(19) United States
(12) Patent Application Publication
(10) Pub. No.: US 2008/0176341 A1
(43) Pub. Date: Jul. 24, 2008

(54) Method for Suppressing Intermolecular Nonspecific Interaction and for Intensifying Intermolecular Specific Interaction on Metal Surface

(75) Inventors: Akito Tanaka, Ibaraki (JP); Tomohiro Terada, Ibaraki (JP); Tsuruki Tamura, Tokyo (JP); Takaaki Shiyama, Osaka (JP); Akira Yamazaki, Osaka (JP); Minoru Furuya, Tokyo (JP); Masayuki Haramura, Kanagawa (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Reverse Protemics Research Institute Co., Ltd., TOKYO (JP)

(21) Appl. No.: 11/573,868

(22) PCT Filed: Aug. 19, 2004
(86) PCT No.: PCT/JP04/12218

§ 371 Date: May 30, 2007

(51) Int. Cl. G01N 33/553

(52) U.S. Cl. 436/525

(57) ABSTRACT

The present invention provides a method of searching for a target molecule for a ligand immobilized on a metal surface or analyzing the interaction between a ligand and a target molecule, characterized in that the immobilization of the ligand on the metal surface via a hydrophilic spacers, and the method eliminates or suppresses a nonspecific interaction that prevents analysis of intermolecular interaction on a metal surface, and can intensify specific intermolecular interactions.

### TECHNICAL FIELD

The present invention relates to a basic technology in intermolecular interactions using a solid phase carrier. More specifically, the present invention relates to a technology to immobilize a molecule to be analyzed onto a metal surface, and to measure and analyze the intermolecular interaction on the metal surface by making use of the interaction, whereby a molecule exhibiting a specific interaction with the molecule to be analyzed is selected and purified or the specific interaction between the molecules is analyzed.

### BACKGROUND ART

In recent years, attempts to search a molecule that exhibits a specific interaction with a particular molecule using a technique based on intermolecular interactions, or research to investigate intermolecular interactions in detail, has been actively conducted. This is specifically represented by research wherein one molecule of the combination of low molecule-low molecule, low molecule-high molecule, or high molecule-high molecule is immobilized onto a solid phase carrier and the interaction between the two molecules is measured, or research wherein a desired target molecule (a molecule that exhibits a specific interaction with a molecule immobilized onto a solid phase carrier) is purified on the basis thereof. As examples of various techniques based on intermolecular interactions, 1) target research using an affinity resin for the latter case, and 2) a method that applies surface plasmon resonance (Surface Plasmon Resonance: SPR) for the former case, are known well. As examples of 1), the discovery of FKBP (FK506 binding proteins), which is the immunosuppressant FK506-binding proteins, using an affinity resin by Professor Schreiber in 1989 (discovery of FKBP12 as a protein that binds to FK506 in cells; for example, Nature (UK), vol. 341, p 758-760, Oct. 26, 1989), the subsequently done discovery of calcineurin inhibitory action in the pharmacological action mechanism of FK506 by an FK506—FKBP complex (for example, Cell (USA), vol. 66, No. 4 p 807-815, Aug. 23, 1991), the discovery of HDAC as a target protein for the anticancer agent Trapoxin (for example, Science (USA), vol. 272, p 408-411, Apr. 19, 1996) and the like are known well. Also, as an example of 2), BIACORE (trade name), which employs a gold foil as a solid phase carrier and enables an extensive investigation of an interaction between a compound or a protein and the like and a protein and the like that specifically interacts therewith, is known well.

However, to date, in the above-described techniques, the presence of a nonspecific intermolecular interaction that hampers the selection and purification of a desired molecule based on a specific intermolecular interaction has been posing such problems as 1) in target search using an affinity resin, a nonspecific protein that masks a specific protein during analysis of a protein bound to the affinity resin using SDS gel and the like exists and makes the detection of the specific protein difficult, or 2) in analysis using BIACORE and the like, the presence of a major peak resulting from nonspecific protein adsorption makes the distinguishing of a peak due to specific protein binding difficult. Although these problems have empirically been considered as being caused by the solid phase carrier, which is an important basic technology, specifically by a surface property of the solid phase carrier, it remains yet to be known clearly which property is the causal factor for a nonspecific interaction, and how to efficiently suppress such a nonspecific intermolecular interaction. For example, some resins such as TentaGel (Fluka Company, Cat. No=86364) have a PEG spacer, are chemically and physically stable, and are also used as resins for affinity chromatography (e.g., Thorpe DS, Walle S., Combinatorial chemistry defines general properties of linkers for the optimal display of peptide ligands for binding soluble protein targets to TentaGel microscopic beads., Biochem Biophys Res Commun 2000 Mar. 16; 269(2):591-5), but basic technologies such as those concerning the identity of the structure that contributes to suppression of a nonspecific interaction and the way of the contribution remain unknown, and there is no sufficient information on to which extent the nonspecific interaction is suppressed or whether or not these resins serve well as affinity resins. As such PEG spacers, TentaGel, which is described above, and ArgoGel (Argonaut Company) are commercially available. Their structures are as follows

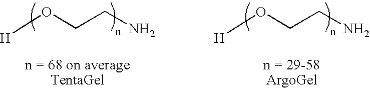

Also, resins comprising a sugar derivative having hydrophilic nature (e.g., AffiGel (AffiGel; Bio-Rad Company, Cat. No=153-2401), a Sepharose derivative (Pharmacia Company, ECH Sepharose 4B, Cat. No=17-0571-01) and the like are known) exhibit minor nonspecific intermolecular interactions, but they are physically and chemically unstable because of their identity as sugar derivatives and their use is limited.

If it is possible to artificially suppress nonspecific interactions in the above-described techniques based on intermolecular interactions, it is considered that the necessity of determining whether the results obtained are due to specific protein binding or nonspecific protein adsorption will be obviated, the frequency of research interruption due to the substantial inability to differentiate both thereof will decrease, and the consumption of protein and the like used will be significantly reduced, so that significant cost reductions in terms of time and labor, and the like will increase the applicability of these techniques.

The present inventors have already found heretofore that the presence of a hydrophilic spacer for immobilization of a ligand onto a solid phase carrier such as a resin and the like reduces a nonspecific interaction between the immobilized ligand molecule and/or the resin per se, and a molecule not specific to the ligand (WO2004/025297). However, such method is based on the mechanism of improving an S/N ratio by mainly suppressing a nonspecific intermolecular interaction, and there still is a demand for a method for intensifying the specific intermolecular interaction itself.

The present invention aims at provision of a method for eliminating or suppressing a nonspecific interaction that prevents analysis of intermolecular interaction particularly on a metal surface, and further aims at provision of a method of purifying or analyzing a target molecule that specifically interacts with a ligand immobilized on a metal surface, while utilizing the method.

### DISCLOSURE OF THE INVENTION

In view of the above-mentioned object, the present inventors have conducted various studies and surprisingly found that introduction of a hydrophilic spacer into a solid phase carrier not only affords an action to suppress a nonspecific interaction but also enhances a specific interaction, particularly when a metal is used as a solid phase carrier. Furthermore, they have succeeded in more accurately searching a target of a ligand, analyzing a specific interaction between a ligand and a target molecule and the like, which resulted in the completion of the present invention.

That is, the present invention is as follows.

[1] A method of suppressing a nonspecific interaction between a ligand and/or a metal surface and a molecule other than a target molecule, which comprises, in a process of immobilizing the ligand onto the metal surface and analyzing a specific interaction on the metal surface between the ligand and the target molecule thereof, a treatment to reduce the hydrophobic property of the metal surface.
[2] A method of intensifying a specific interaction between a ligand and a target molecule, which comprises, in a process of immobilizing the ligand onto a metal surface and analyzing a specific interaction on the metal surface between the ligand and the target molecule of the ligand, a treatment to reduce the hydrophobic property of the metal surface.
[3] A method of suppressing a nonspecific interaction between a ligand and/or a metal surface and a molecule other than a target molecule and intensifying a specific interaction between the ligand and the target molecule, which comprises, in a process of immobilizing the ligand onto the metal surface and analyzing a specific interaction on the metal surface between the ligand and the target molecule of the ligand, a treatment to reduce the hydrophobic property of the metal surface.
[4] A method of suppressing a nonspecific interaction between a ligand and/or a metal surface and a molecule other than a target molecule, which comprises, in a process of immobilizing the ligand onto the metal surface and selecting a target molecule using a specific interaction on the metal surface between the ligand and the target molecule thereof, a treatment to reduce the hydrophobic property of the metal surface.
[5] A method of intensifying a specific interaction between a ligand and a target molecule, which comprises, in a process of immobilizing the ligand onto the metal surface and selecting the target molecule using a specific interaction on the metal surface between the ligand and the target molecule thereof, a treatment to reduce the hydrophobic property of the metal surface.
[6] A method of suppressing a nonspecific interaction between a ligand and/or a metal surface and a molecule other than a target molecule and intensifying a specific interaction between the ligand and the target molecule, which comprises, in a process of immobilizing the ligand onto the metal surface and selecting the target molecule using a specific interaction on the metal surface between the ligand and the target molecule thereof, a treatment to reduce the hydrophobic property of the metal surface.
[7] The method of any one of [1]-[6] above, wherein the treatment to reduce the hydrophobic property of the metal surface is to introduce, at the time of immobilization of the ligand onto the metal surfacer a hydrophilic spacer therebetween.
[8] The method of [7] above, wherein the hydrophilic spacer has at least any of the following characteristics while in a state bound to the metal surface and the ligand:
(i) the number of hydrogen bond acceptor is 6 or more,
(ii) the number of hydrogen bond donor is 5 or more,
(iii) the total number of hydrogen bond acceptor and hydrogen bond donor is 9 or more.
[9] The method of [8] above, wherein said hydrophilic spacer further has one or more carbonyl groups in the molecule thereof.
[10] The method of [8] or [9] above, further characterized in that said hydrophilic spacer does not have a functional group that becomes positively or negatively charged in an aqueous solution.
[11] A method of immobilizing a ligand onto a metal surface and analyzing a specific interaction on the metal surface between so the ligand and a target molecule thereof, which comprises suppressing a nonspecific interaction between the ligand and/or the metal surface and a molecule other than the target molecule by a treatment to reduce the hydrophobic property of the metal surface.
[12] A method of immobilizing a ligand onto a metal surface and analyzing a specific interaction on the metal surface between the ligand and a target molecule thereof, which comprises intensifying a specific interaction between the ligand and the target molecule by a treatment to reduce the hydrophobic property of the metal surface.
[13] A method of immobilizing a ligand onto a metal surface and analyzing a specific interaction on the metal surface between the ligand and a target molecule thereof, which comprises suppressing a nonspecific interaction between the ligand and/or the metal surface and a molecule other than the target molecule and intensifying a specific interaction between the ligand and the target molecule, by a treatment to reduce the hydrophobic property of the metal surface.
[14] A method of immobilizing a ligand onto a metal surfacer and selecting a target molecule using a specific interaction on the metal surface between the ligand and the target molecule thereof, which comprises suppressing a nonspecific interaction between the ligand and/or the metal surface and a molecule other than the target molecule by a treatment to reduce the hydrophobic property of the metal surface.
[15] A method of immobilizing a ligand onto a metal surface, and selecting a target molecule using a specific interaction on the metal surface between the ligand and the target molecule thereof, which comprises intensifying a specific interaction between the ligand and the target molecule by a treatment to reduce the hydrophobic property of the metal surface.
[16] A method of immobilizing a ligand onto a metal surface, and selecting a target molecule using a specific interaction on the metal surface between the ligand and the target molecule thereof, which comprises suppressing a nonspecific interaction between the ligand and/or the metal surface and a molecule other than the target molecule and intensifying a specific interaction between the ligand and the target molecule, by a treatment to reduce the hydrophobic property of the metal surface.
[17] The method of any one of [11]-[16] above, wherein the treatment to reduce the hydrophobic property of the metal surface is to introduce, at the time of immobilization of the ligand onto the metal surface, a hydrophilic spacer therebetween.
[18] The method of [17] above, wherein the hydrophilic spacer has at least any of the following characteristics while in a state bound to the metal surface and the ligand:
(i) the number of hydrogen bond acceptor is 6 or more,
(ii) the number of hydrogen bond donor is 5 or more,
(iii) the total number of hydrogen bond acceptor and hydrogen bond donor is 9 or more.
[19] The method of [18] above, wherein said hydrophilic spacer further has one or more carbonyl groups in the molecule thereof.
[20] The method of [18] or [19] above, further characterized in that said hydrophilic spacer does not have a functional group that becomes positively or negatively charged in an aqueous solution.
[21] A screening method for a target molecule having a specific interaction with a ligand, comprising at least the following steps:
(i) immobilizing the ligand onto a metal surface via a hydrophilic spacer,
(ii) contacting a sample that contains or does not contain the target molecule with the metal surface with the ligand immobilized thereon obtained in (i) above,
(iii) identifying and analyzing a molecule that has exhibited or has not exhibited a specific interaction with the ligand, and
(iv) judging a molecule that exhibits a specific interaction with the ligand as the target molecule on the basis of the analytical results obtained in (iii) above.
[22] The method of [21] above, wherein the hydrophilic spacer has at least any of the following characteristics while in a state bound to the metal surface and the ligand:
(i) the number of hydrogen bond acceptor is 6 or more,
(ii) the number of hydrogen bond donor is 5 or more,
(iii) the total number of hydrogen bond acceptor and hydrogen bond donor is 9 or more.
[23] The method of [22] above, wherein said hydrophilic spacer further has one or more carbonyl groups in the molecule thereof.
[24] The method of [22] or [23] above, further characterized in that said hydrophilic spacer does not have a functional group that becomes positively or negatively charged in an aqueous solution.
[25] The method of any one of [7]-[10], [17]-[24] above, wherein the hydrophilic spacer has at least one partial structure represented by any one formula selected from the group consisting of Formulas (Ia)-(Ie) below:

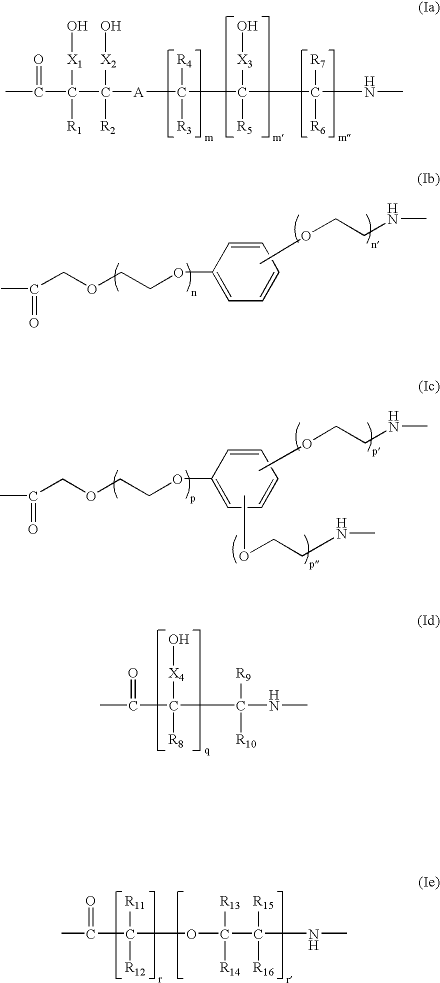

### (In Formula (Ia),

A is an appropriate joining group,
X₁-X₃ are the same or different and each is a single bond or a methylene group that may be substituted by a linear or branched alkyl group having 1-3 carbon atoms,
R₁-R₇ are the same or different and each is a hydrogen atom, a linear or branched alkyl group having 1-3 carbon atoms, —CH₂OH or a hydroxyl group,
m is an integer of 0-2, m′ is an integer of 0-10, m″ is an integer of 0-2,
when a plurality of R₃-R₇ units exist, they may be the same or different, when a plurality of X₃ units exist, they may be are the same or different;

### in Formula (Ib),

n and n′ are the same or different and each is an integer of 1-1000;

### in Formula (Ic),

p, p′ and p″ are the same or different and each is an integer of 1-1000;

### in Formula (Id),

X₄ is a single bond or a methylene group that may be substituted by a linear or branched alkyl group having 1-3 carbon atoms,
R₈-R₁₀ are the same or different and each is a hydrogen atom, a linear or branched alkyl group having 1-3 carbon atoms, —CH₂OH or a hydroxyl group,
q is an integer of 1-7,
when a plurality of R₈ units exist, they may be the same or different, when a plurality of X₄ units exist, they may be the same or different;

### in Formula (Ie),

R₁₁-R₁₆ are the same or different and each is a hydrogen atom, a linear or branched alkyl group having 1-3 carbon atoms, —CH₂OH or a hydroxyl group,
r is an integer of 1-10, r′ is an integer of 1-50,
when a plurality of R₁₁-R₁₆ units exist, they may be the same or different).
[26] The method of [25] above, wherein the hydrophilic spacer has two or more partial structures represented by any one formula selected from the group consisting of Formulas (Ia)-(Ie).
[27] A solid phase carrier with a ligand immobilized thereon, which is a metal and has a hydrophilic spacer between the metal and the ligand.
[28] The solid phase carrier of [27] above, wherein the hydrophilic spacer has at least one partial structure represented by any one formula selected from the group consisting of Formulas (Ia)-(Ie).
[29] The solid phase carrier of [27] or [28] above, wherein the metal is gold.
[30] A method of confirming introduction of a hydrophilic spacer between a ligand and a metal surface, which comprises, in a step of introducing the hydrophilic spacer between them during immobilization of the ligand onto the metal surface, detecting a leaving group produced by elimination of a protecting group derived from the hydrophilic spacer.
[31] The method of [30] above, wherein the leaving group is detected by mass analysis.
[32] The method of [30] above, wherein the protecting group is a 9-fluorenylmethyloxycarbonyl group.

### BRIEF DESCRIPTION OF THE DRAWINGS

FIG. 1 is a photograph of the electrophoresis showing the comparison results between the case wherein a ligand is immobilized onto a gold film surface via a hydrophilic spacer (Production Example 11) and the case wherein a ligand is immobilized onto a gold film surface without a hydrophilic spacer (Reference Example 1), with respect to the adsorption of a nonspecific binding protein to the gold film surface (ligand) and binding thereto of a specific protein.

### DETAILED DESCRIPTION OF THE INVENTION

The present invention is based on the finding that nonspecific intermolecular interactions (e.g., represented by nonspecific adsorption of a protein to a solid phase carrier), which have been viewed as being problematic in the technology to analyze and utilize a specific intermolecular interaction, are due to the hydrophobic interaction between the solid phase surface in a solid phase carrier and a molecule such as of a protein. Particularly, the present invention also provides a method of suppressing nonspecific adsorption of various molecules to a metal surface and increasing the amount of specific adsorption, by a treatment to reduce the hydrophobic property of the metal surface.

In the present specification, the hydrophobic property can generally be represented by a hydrophobicity parameter, and can, for example, be represented by partition coefficient, specifically by LOGP. In calculating LOGP, CLOGP (a predicted value obtained using a software program for estimating hydrophobicity parameters of a compound by means of a computer; can be calculated using, for example, Corwin/Leo's program (CLOGP, Daylight Chemical Information System Co., Ltd)) and the like are conveniently utilized, but the hydrophobicity parameter is not limited to CLOGP. In the present invention, as the tendency for hydrophobicity increases qualitatively, nonspecific interactions increase. For example, referring to CLOGP, the greater the CLOGP is, the higher the hydrophobicity is; the increase in CLOGP correlates to the increase in a nonspecific interaction (e.g., nonspecific adsorption of a protein to a metal surface). Here, a change in a hydrophobicity parameter can, for example, be performed by changing the ligand to be immobilized onto the metal surface to one having various values (e.g., CLOGP) of hydrophobicity parameter, and it is also possible to moderate or reduce the hydrophobic property of the metal surface by introducing a hydrophilic spacer between the metal surface and the ligand.

Introducing the spacer is a preferred embodiment in cases wherein it is necessary to immobilize onto a metal surface a ligand expected to have a high CLOGP value; a case wherein a hydrophilic spacer is used as a means of suppressing a nonspecific intermolecular interaction is hereinafter described in detail.

The present invention provides a technology to analyze the interaction between a molecule immobilized onto a metal surface (in the present specification, also defined as ligand) and a molecule that exhibits a specific interaction with the molecule described above (in the present specification, also defined as target molecule), and a technology to identify and select the target molecule on the basis of such analysis. In the present specification, the terms ligand and target molecule are intended to mean a combination of members that exhibit a specific intermolecular interaction with each other, and their designations are variable depending on which member of the combination to immobilize as the ligand onto the solid phase and leave the other member as the target molecule, that is, which member to immobilize onto the solid phase. There can be more than one kind of the target molecule that exhibits a specific interaction with the ligand, and likewise there can be more than one kind of the ligand that exhibits a specific interaction with the target molecule. In the present specification, the terms ligand and target molecule do not refer to particular molecules but are intended to mean individual molecules that exhibit a specific interaction with each other.

A “specific interaction” is a characteristic action to specifically recognize, and bind to, a particular ligand (a particular target molecule) only; the relation of a specific receptor to an agonist or an antagonist, the relation of an enzyme to a substrate, and, for example, the relation of an FK506-binding protein (target molecule) to FK506 (ligand), the relation of a steroid hormone receptor to a steroid hormone (e.g., dexamethasone and glucocorticoid receptor), the relation of HDAC to the anticancer agent trapoxin, and the like apply to a “specific interaction”. On the other hand, a “nonspecific interaction” refers to an action the subjects of binding by which encompass a broad range of molecules and are not limited to particular molecules, and which produces a situation that is variously changeable depending on reaction conditions; in the present invention, this term means an unparticular intermolecular action to bind or adsorb to the ligand on a solid phase or the solid phase carrier surface. A “nonspecific interaction” is risky in that the binding based on a “specific interaction” is possibly overlooked as it hampers, or is confused with, the binding of the ligand and the target molecule based on a “specific interaction”.

In the present invention, “to analyze a specific interaction” is to obtain the extent of the specific interaction between a ligand and a target molecule as interaction information, which can, for example, be obtained as numerical values of Kd (dissociation rate constant), Ka (association rate constant) and the like. In the present invention, “selection” is intended to mean determining whether or not the molecule in question exhibits a specific interaction with the ligand on the basis of the above-described interaction information, and identifying the target molecule.

In the present invention, a treatment to reduce the hydrophobic property of the metal surface is essential. As an example of the treatment, a method of introducing a hydrophilic spacer, at the time of immobilization of the ligand to the metal surface, therebetween, can be mentioned. By introducing a hydrophilic spacer, the hydrophobic property of the metal surface is altered, so that a nonspecific interaction can be suppressed, as well as a specific interaction can be intensified. By using such a means of suppressing a nonspecific interaction, called a hydrophilic spacer, introduced between the metal surface and the ligand, it is possible to identify and select a molecule (target molecule) that exhibits a specific interaction with the ligand, and to accurately measure the interaction therebetween.

The metal as a solid phase carrier to be used in the present invention includes various kinds generally used in this field, which may be specifically gold, silver, iron, silicone and the like. These may have any shape, which is appropriately determined according to the kind of the above-mentioned metal, the analysis to be performed thereafter of the interaction between a ligand and a target molecule, and a method to be used for the identification and selection of the target molecule. For example, plates, thin films, threads, coils and the like can be mentioned; metallic thin films can be preferably used as carriers for BIACORE and the like by surface plasmon resonance.

Although the metal used in the present invention, as described above, is not subject to limitation as to the kind and form thereof, one having a structural hindrance such that the ligand is not immobilizable thereon or the ligand is immobilizable thereon but cannot exhibit a specific interaction with the target molecule, of course, is undesirable for embodying the present invention because it complicates the operation due to the necessity of an additional step or is unusable in some cases.

In the present invention, “hydrophilic spacer” refers to a substance that is introduced to become a group that interlies between a metal surface and a ligand at the time of immobilization of the ligand onto the metal surface, and is hydrophilic. Degrees of hydrophilicity are described below. Here, “a spacer interlies” means that the spacer is present between a functional group in the solid phase and a functional group in the ligand. The spacer binds to the functional group in the solid phase at one end and binds to the functional group in the ligand at the other end.

Also, the hydrophilic spacer may be one obtained by sequentially binding and polymerizing 2 or more compounds, as long as it is capable of eventually functioning as a group that interlies between the metal surface and the ligand. Preferably, the hydrophilic spacer is obtained by a polymerization reaction of a unit compound. The process to bind or polymerize 2 or more compounds is preferably conducted on a metal surface. Binding of the metal surface and the hydrophilic spacer, binding of the hydrophilic spacer and the ligand, and binding and polymerization of individual components that constitute the hydrophilic spacer are based on a covalent bond or a non-covalent bond, such as an amide bond, a Schiff base, a C—C bond, an ester bond, a hydrogen bond or a hydrophobic interaction, all of which are formed using materials and reactions known in the art.

In the present invention, the hydrophilic spacer introduced between a metal surface and a ligand as a means of reducing the hydrophobic property of the metal surface is not subject to limitation, as long as it alters the hydrophobic property of the metal surface to eliminate or suppress the nonspecific interaction and to intensify the specific interaction, and is preferably a compound having the number of hydrogen bond acceptor (HBA; hydrogen bond acceptor) of 6 or more, or the number of hydrogen bond donor (HBD; hydrogen bond donor) of 5 or more, or the total number of HBA and HBD per spacer molecule of 9 or more while in a state bound to metal surface and the ligand (a hydrophilic spacer in this state is hereinafter referred to as “hydrophilic spacer moiety” for convenience). Also, the hydrophilic spacer may be a compound that meets two or all of these conditions. Particularly preferably, the number of HBA is 7 or more and the number of HBD is 6 or more.

Here, the number of hydrogen bond acceptor (the number of MBA) is the total number of nitrogen atoms (N) and oxygen atoms (O) contained, and the number of hydrogen bond donor (the number of HBD) is the total number of NH and OH contained (for example, Advanced Drug Delivery Reviews, Netherland, 23 (1997) p. 3-25).

For immobilization of a ligand on the metal surface, a method comprising adsorbing a thiol compound or disulfide compound onto a metal surface to form a Self-Assembled Monolayer (SAM), and immobilizing the ligand on its terminal is generally employed (see Dojin News No. 91 p 3 (1999)). By forming a SAM on a metal surface and immobilizing the ligand on the surface via a functional group in the SAM, an interaction between a ligand and a target molecule can be detected by a gold-modified electrode, surface plasmon resonance, Quartz Crystal Microbalance (QCM) and the like (specifically, detected based on the changes in electric current, reflection angle or vibration frequency, respectively). For example, when gold is used as a metal to be the solid phase carrier, alkanethiol is used as a thiol compound.

In the present invention, therefore, a connection part minimum required for binding a ligand to a metal surface (specifically, a part derived from the above-mentioned thiol compound or disulfide compound) is not included in the hydrophilic spacer of the present invention, even if it is a group intervening between a metal surface and a ligand, and therefore, is not included in the number of HBA and the number of HBD. To facilitate binding of a ligand to a hydrophilic spacer, moreover, it is possible to bind or introduce an optional group to or into the ligand, in between the ligand and the hydrophilic spacer, before binding to the hydrophilic spacer. However, since they are appropriately selected according to the ligand, and considered to less contributing to the mitigation of the hydrophobic property of the metal surface, N, O, NH and OH contained in such group are not counted in the number of HBD and the number of HBA in the present invention. For introduction of the optional group in between the ligand and the hydrophilic spacer, the aforementioned various covalent bonds and noncovalent bonds can be utilized, using materials and reactions known in this field.

Under the circumstances of the invention of the present application, nonspecific interactions cannot be fully suppressed and specific interactions cannot be sufficiently intensified, unless at least one, preferably 2 or more, of the conditions of the number of HBA of 6 or more (preferably 7 or more), the number of HBD of 5 or more (preferably 6 or more), and the total number of HBA and HBD of 9 or more, are met. Therefore, in the hydrophilic spacer of the invention of the present application, “hydrophilic” means that the above-described requirements are met. In the present invention, the upper limit of the number of HBD or the number of HBA of the hydrophilic spacer is not subject to limitation, as long as the spacer is hydrophilic and capable of suppressing a nonspecific interaction; by appropriately repeating a polymerization reaction and the like, a spacer having an extremely high hydrophilicity can be obtained. Also, the spacer may be a high-molecular substance such as a protein; from this viewpoint, the upper limit should be at a value of 50,000 or so in all cases.

In the present invention, moreover, a hydrophilic spacer with a physically or chemically unstable compound, such as a sugar derivative or a sepharose derivative, as a basic skeleton, is not preferable for use, even if it satisfies the degree of “hydrophilicity” defined above, since it may be too unstable to stand the immobilization of a ligand and subsequent various treatments. Specifically, carboxymethyldextran conventionally used for immobilization of a ligand on a gold thin film is not included in the hydrophilic spacer to be used in the present invention.

Furthermore, the hydrophilic spacer used in the present invention is preferably one that does not exhibit a nonspecific interaction (e.g., protein adsorption to the spacer and the like) per se. Specifically, it is preferable that the spacer does not have a functional group that becomes positively or negatively charged in an aqueous solution; as the functional group, an amino group (but excluding cases wherein a functional group that attenuates the basicity of the amino group (e.g., a carbonyl group, a sulfonyl group) is bound to the amino group), a carboxyl group, a sulfuric acid group, a nitric acid group, a hydroxamic acid group and the like can be mentioned. Here, “in an aqueous solution” specifically refers to an environment wherein the process to analyze the interaction between a ligand and a target molecule on a metal surface, the process to select the target molecule, or a binding reaction (a reaction based on a specific interaction) of the ligand and the target molecule performed to screen for the target molecule is conducted, and whereunder the hydrophilic spacer ionizes when having a functional group that becomes positively or negatively charged. Such conditions are, for example, in an aqueous solution, pH 1-11, temperature 0° C.-100° C., preferably pH nearly neutral (pH 6-8), about 4° C. to about 40° C. or so.

Furthermore, the hydrophilic spacer used in the present invention preferably has 1 or more carbonyl groups in the molecule thereof, as understood from the various structures or compounds described below as preferable examples of the hydrophilic spacer.

For example, the hydrophilic spacer used in present invention is a compound that has at least one partial structure represented by any one formula selected from the group consisting of Formulas (Ia)-(Ie) below.

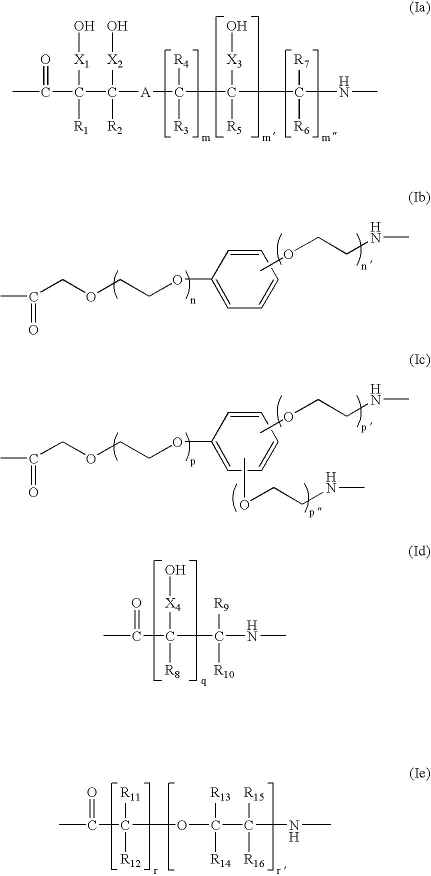

### (In Formula (Ia),

A is an appropriate joining group,
X₁-X₃ are the same or different and each is a single bond or a methylene group that may be substituted by a linear or branched alkyl group having 1-3 carbon atoms,
R₁-R₇ are the same or different and each is a hydrogen atom, a linear or branched alkyl group having 1-3 carbon atoms, —CH₂OH or a hydroxyl group,
m is an integer of 0-2, m′ is an integer of 0-10, m″ is an integer of 0-2,
when a plurality of R₃-R₇ units exist, they may be the same or different, when a plurality of X₃ units exist, they may be the same or different;

### in Formula (Ib),

n and n′ are the same or different and each is an integer of 1-1000;

### in Formula (Ic),

p, p′ and p″ are the same or different and each is an integer of 1-1000;

### in Formula (Id),

X₄ is a single bond or a methylene group that may be substituted by a linear or branched alkyl group having 1-3 carbon atoms,
R₈-R₁₀ are the same or different and each is a hydrogen atom, a linear or branched alkyl group having 1-3 carbon atoms, —CH₂OH or a hydroxyl group,
q is an integer of 1-7,
when a plurality of R₈ units exist, they may be the same or different, when a plurality of X₄ units exist, they may be the same or different;

### in Formula (Ie),

R₁₁-R₁₆ are the same or different and each is a hydrogen atom, a linear or branched alkyl group having 1-3 carbon atoms, —CH₂OH or a hydroxyl group,
r is an integer of 1-10, r′ is an integer of 1-50,
when a plurality of R₁₁-R₁₆ units exist, they may be the same or different).

In the present specification, referring to the definitions for individual groups, the “appropriate joining group” is not subject to limitation, as long as it is capable of joining mutually adjoining sites, and specifically the following groups are used.

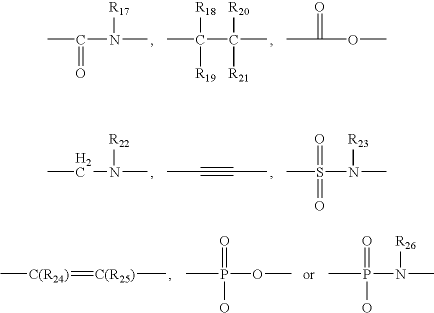

(in the formulas, R₁₇ is a hydrogen atom or a linear or branched alkyl group having 1-3 carbon atoms, R₁-R₂, are the same or different and each is a hydrogen atom, a linear or branched alkyl group having 1-3 carbon atoms, —CH₂OH or a hydroxyl group, R₂₂-R₂₆ are the same or different and each is a hydrogen atom or a linear or branched alkyl group having 1-3 carbon atoms (the alkyl group may be substituted by a hydrophilic substituent such as a hydroxyl group, a carboxylic acid group, or an amino group))

In the present specification, referring to the definitions for individual groups, as examples of the “linear or branched alkyl group having 1-3 carbon atoms”, a methyl is group, an ethyl group, a propyl group, an isopropyl group and the like can be mentioned.

In the present specification, “a methylene group that may be substituted by a linear or branched alkyl group having 1-3 carbon atoms”, is intended to mean an unsubstituted methylene group and a methylene group substituted by 1 or 2 of the above-described linear or branched alkyl groups having 1-3 carbon atoms.

The hydrophilic spacer of the present invention may have two or more of the above-described partial structure; in that case, the partial structures may be represented by the same formula or represented by different formulas.

At least one kind of the above-described hydrophilic spacer is immobilized onto a metal surface. The number of spacers on the metal surface is not subject to limitation, and can be appropriately determined by those skilled in the art according to the kind and amount of the ligand, the kind and amount of the target molecule, and the kind and characteristic of the spacer used, and needs not be determined, provided that the desired intermolecular interaction can be detected. Usually, the spacer is immobilized using an excess amount thereof relative to the metal used as a solid phase carrier and is the ligand. The hydrophilic spacers that have not bound to the metal surface can easily be removed from the reaction system by a treatment such as washing.

In the present invention, the ligand to be immobilized onto a metal surface is not subject to limitation, and may be a known compound or a novel compound that will be developed in the future. Also, the ligand may be a low molecular compound or a high molecular compound. Here, a low molecular compound refers to a compound having a molecular weight of less than 1000 or so; for example, an organic compound commonly usable as a pharmaceutical, a derivative thereof, and an inorganic compound can be mentioned; specifically, a compound produced by making use of a method of organic synthesis and the like, a derivative thereof, a naturally occurring compound, a derivative thereof, a small nucleic acid molecule such as a promoter, various metals, and the like can be mentioned; and desirably, an organic compound that can be used as a pharmaceutical, a derivative thereof, or a nucleic acid molecule can be referred to. Also, as the high molecular compound, a compound having a molecular weight of 1000 or more or so, which is a protein, a polynucleic acid, a polysaccharide, or a combination thereof, and the like can be mentioned, and a protein is desirable. These low molecular compounds or high molecular compounds are commercially available if they are known compounds, or can be obtained via steps such as of collection, production and purification according to various publications. These may be of natural origin, or may be prepared by gene engineering, or may be obtained by semi-synthesis and the like.

In the present invention, a process to select a target molecule on the basis of the specific interaction with the above-described ligand on a metal surface having the ligand immobilized thereon is necessary. Therefore, the target molecule is not subject to limitation, as long as it specifically interacts with the ligand, and is expected to be a known compound in some cases or a novel substance in other cases. The target molecule may be a low molecular compound or a high molecular compound. When the target molecule is a low molecular compound, the target molecule can be selected on the basis of the specific interaction with the ligand that is a low molecular compound, which is a low molecular compound-low molecular compound interaction, or on the basis of the specific interaction with the ligand that is a high molecular compound, which is a high molecular compound-low molecular compound interaction. Also, when the target molecule is a high molecular compound, the target molecule can be selected on the basis of the specific interaction with the ligand that is a low molecular compound, which is a low molecular compound-high molecular compound interaction, or on the basis of the specific interaction with the ligand that is a high molecular compound, which is a high molecular compound-high molecular compound interaction. A preferable combination of the ligand and the target molecule is the combination of a low molecular compound and a high molecular compound, or the combination of a high molecular compound and a high molecular compound.

Analysis of the interaction of the ligand with the target molecule and selection of the target molecule are conveniently conducted on a metal surface which is a solid phase. When a candidate substance is anticipated as the target molecule, it is possible to bring the candidate substance alone into contact with the above-described ligand immobilized on the metal surface, measure the interaction therebetween, and determine whether or not the candidate substance is the target molecule; usually, by bringing a sample that contains a plurality of substances (a high molecular compound and/or a low molecular compound) into contact with the ligand, and measuring the presence or absence of an interaction between each of the plurality of substances (the high molecular compound and/or the low molecular compound) and the ligand and the extent of the interaction, whether or not each substance is the target molecule is determined and the target molecule is selected. Here, the sample that contains a plurality of substances may consist essentially of known compounds, may contain some novel compounds, and may consist essentially of novel compounds. However, according to search of target molecules for ligands, or recent advances in proteome analysis, it is desirable that the sample be a mixture essentially of compounds of known structures. As the sample consisting essentially of known compounds, a mixture of proteins prepared by gene engineering using Escherichia coli and the like, and the like can be mentioned; as the sample that contains some novel compounds, a cell or tissue extract (Lysate) can be mentioned; as the sample that consists essentially of novel compounds, a mixture of novel proteins whose functions and structures are yet unknown, or newly synthesized compounds and the like, can be mentioned. When the sample is a mixture, especially when it contains known compounds, the contents of these compounds in the sample may optionally be set at desired levels in advance, From the viewpoint of searching a target molecule for a ligand, target molecule to be selected is preferably a low molecular compound or a high molecular compound, and for searching a target molecule in the body of an animal such as a human, the target molecule is preferably a high molecular compound.

The present invention provides, using the above-described ligand immobilized on the metal surface, a method of screening for a target molecule that exhibits a specific interaction with the ligand. The screening method includes at least the following steps. Note that the respective definitions for the ligand, the target molecule, the metal (metal surface), and the hydrophilic spacer in this screening method are as described above.

(1) A Step of Immobilizing the Ligand onto a Metal Surface Via a Hydrophilic Spacer.

This step comprises binding of the ligand and the hydrophilic spacer and binding of the hydrophilic spacer and the metal surface. It is possible to bind the hydrophilic spacer to the ligand and then bind the complex thereof to the metal surface, and also possible to bind the ligand after the hydrophilic spacer is bound to the metal surface; whether or not the ligand has been immobilized onto the metal surface can be confirmed by utilizing a reaction based on a particular structure or substituent and the like contained in the ligand or an optionally chosen group that has been bound and introduced to the ligand in advance, and the like. For example, a method comprising detecting or measuring a leaving group produced during elimination of a 9-fluorenylmethyloxycarbonyl group (Fmoc group), which is an amino-protecting group in a ligand or hydrophilic spacer, and the like can be employed. Each binding is performed by utilizing a reaction in common use in the art. As a convenient and accurate means, a method utilizing an amide bond formation reaction can be mentioned. This reaction can, for example, be performed according to “Pepuchido Gousei no Kiso to Jikken” (ISBN 4-621-02962-2, Maruzen, first edition issued in 1985). Regarding the reagents and solvents used in each reaction, those in common use in the relevant field can be utilized, and are appropriately selected according to the binding reaction employed.

(2) A Step of Contacting a Sample that Contains or does not Contain the Target Molecule with the Metal with the Ligand Immobilized Thereon Obtained in (1) Above.

The sample used in this step is one containing a plurality of substances as described above. The mode of embodiment thereof is not subject to limitation, and can be appropriately changed according to the metal used as solid phase carrier and their shape, principles, means and methods to use for the identification or analysis in the subsequent steps (3) and (4). For example, when analysis is performed by BIACORE (trade name) and using a gold thin film on which a ligand has been immobilized, it is preferable that the sample be liquid. In the case of a sample that does not contain the target molecule, identification and analysis of a molecule (a plurality of kinds present in some cases) that has not exhibited a specific interaction with the ligand in step (3) are conducted. In the case of a sample that contains the target molecule, the target molecule (a plurality of kinds present in some cases) that has exhibited a specific interaction with the ligand in step (3) is identified and analyzed. The method of bringing the sample and the metal surface into contact with each other is not subject to limitation, as long as the target molecule in the sample can bind to the ligand immobilized on the metal surface, and can be appropriately changed according to the kinds of metal used and their shape, principles, means and methods to use for the identification or analysis in the subsequent steps (3) and (4). For example, when a gold thin film on which a ligand has been immobilized is used, the treatment includes immersion of the gold thin film in a liquid sample and the like.

(3) A Step of Identifying and Analyzing a Molecule that has Exhibited or has not Exhibited a Specific Interaction with the Ligand.

Although this step can be appropriately changed according to the kinds and shape of the metal used as solid phase carrier and the kinds of the ligand, and the like, it is conducted by various methods in common use in the art to identify a low molecular compound or a high molecular compound. Also, the step can also be performed by a method that will be developed in the future. For example, when a gold thin film on which a ligand has been immobilized is used as a metal having a ligand immobilized on its surface [step (1)], the target molecule is bound to the ligand by the subsequent addition of the sample [step (2)]. It is also possible to dissociate the target molecule bound from the ligand by a treatment such as altering the polarity of the buffer solution or further adding the ligand in excess, and then identify the target molecule, or to extract the target molecule with a surfactant and the like while remaining in a state bound to the ligand on the metal surface, and then identify the target molecule. As the method of identification specifically, known techniques such as electrophoresis, immunoblotting and immunoprecipitation, which employ immunological reactions, chromatography, mass spectrometry, amino acid sequencing, NMR (especially for low-molecules), and reactions utilizing surface plasmon resonance, or combinations of these methods can be used. Although the step of identifying a molecule that does not bind to the ligand can also be conducted in accordance with the above-described method of identifying a molecule that binds to the ligand, it is preferable that a treatment such as concentration or crude purification be conducted in advance before entering the identification step, since a molecule contained in the trough fraction from the column is the subject of identification. On the basis of the data obtained and existing reports, each molecule is identified, and whether or not it is a target molecule for the ligand is determined.

Also, this step may be automated. For example, it is also possible to directly read data on various molecules, which have been obtained by two-dimensional electrophoresis, and identify the molecules on the basis of existing databases.

A general method of producing the hydrophilic spacer of the present invention is described below, but it is obvious to those skilled in the art that the same can also be produced by other methods in common use in the art or combinations thereof.

Note that the abbreviations used in the present specification are as follows.

### ABBREVIATION FORMAL DESIGNATION

Ac Acetyl group
Bn Benzyl group
Bu₃P Tributylphosphine
DMAP Dimethylaminopyridine
DMF Dimethylformamide
EDC 1-[3-(Dimethylamino)propyl]-3-ethylcarbodiimide
Et Ethyl group
Fmoc 9-Fluorenylmethyloxycarbonyl group
Fmoc-OSu 9-Fluorenylmethylsuccinimidylcarbonate
Gold foil Gold film
HOBt 1-Hydroxybenzotriazole
HyT Hydrazinotartaric amide
Me Methyl group
PEG Polyethylene glycol
Ph₃P Triphenylphosphine
PyBOP Benzotriazol-1-yl-oxy-tris-pyrrolidino-phosphonium hexafluorophosphate
TBAF Tetrabutylammonium fluoride
TBDMS tert-Butyldimethylsilyl group
TBDMSOTf Trifluoromethanesulfonic acid t-butyldimethylsilyl group
TBDPS tert-Butyldiphenylsilyl group
TBS tert-Butyldimethylsilyl group
tBu tert-Butyl group
TFA Trifluoroacetic acid
THF Tetrahydrofuran
TMAD N,N,N′,N′-tetramethylazodicarboxamide
Tr Trityl group
Ts Tosyl group (toluenesulfonyl group)
WSC Water-soluble carbodiimide (N-ethyl-N′-(3′-dimethylaminopropyl)carbodiimide)

### Process 1: Production Method (1) for the Hydrophilic Spacer Having a Partial Structure Represented by General Formula (Ia)

(m=1, m′=2, m″=1)

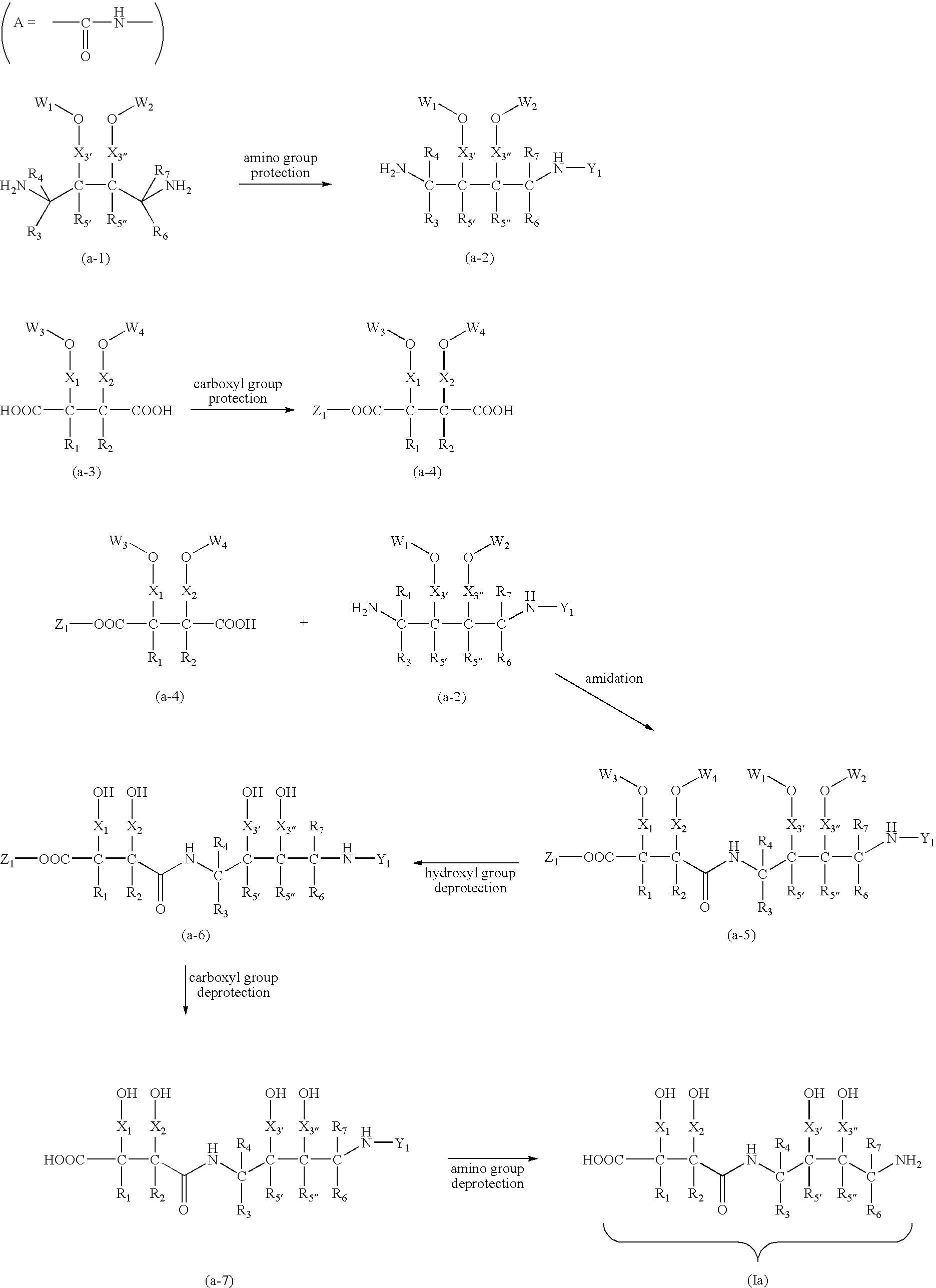

In the formulas, W₁-W₄ are hydroxyl-group-protecting groups, Z₁ is a carboxyl-group-protecting group, and Y₁ is an amino-group-protecting group. X_{3′}, has the same definition as X₃ and X_{3″} has the same definition as X₃. R_{5′}, has the same definition as R₅ and R_{5″} has the same definition as R₅. Also, the definitions for the other individual symbols are as described above.

As the hydroxyl-group-protecting group, an optionally chosen group in common use in the art is used; specifically, alkyl groups such as a tert-butyl group; acyl groups such as an acetyl group, a propionyl group, a pivaloyl group and a benzoyl group; alkoxycarbonyl groups such as a methoxycarbonyl group and a tert-butoxycarbonyl group; aralkyloxycarbonyl groups such as a benzyloxycarbonyl group; arylmethyl groups such as a benzyl group and a naphthylmethyl group; silyl groups such as a trimethylsilyl group, a triethylsilyl group, a tert-butyldimethylsilyl group and a tert-butyldiphenylsilyl group; lower alkoxymethyl groups such as an ethoxymethyl group and a methoxymethyl group, and the like can be mentioned as examples, and preferably, a tert-butyldimethylsilyl group, a tert-butyldiphenylsilyl group, a methoxymethyl group and a tert-butyl group can be mentioned. As the carboxyl-group-protecting group, an optionally chosen group in common use in the art is used; specifically, linear or branched lower alkyl groups having 1-6 carbon atoms, such as a methyl group, an ethyl group, a propyl group, a tert-butyl group, an isobutyl group and an allyl group; aralkyl groups such as a benzyl group; silyl groups such as a tert-butyldimethylsilyl group and a tert-butyldiphenylsilyl group, and the like can be mentioned as examples; preferably, an allyl group, a tert-butyl group, a benzyl group and a tert-butyldiphenylsilyl group can be mentioned. As the amino-group-protecting group, an optionally chosen group in common use in the art is used; specifically, lower alkoxycarbonyl groups such as a tert-butoxycarbonyl group, a methoxycarbonyl group and 9-fluorenylmethyloxycarbony group; aralkyloxycarbonyl groups such as a benzyloxycarbonyl group; aralkyl groups such as a benzyl group; substituted sulfonyl groups such as a benzenesulfonyl group, a p-toluenesulfonyl group and a methanesulfonyl group, and the like can be mentioned as examples, preferably, a tert-butoxycarbonyl group and a benzyloxycarbonyl group can be mentioned.

Amino group protection and deprotection, carboxyl group protection and deprotection, and hydroxyl group deprotection are appropriately performed using known methods and reagents according to the protective group used. In addition, when a compound has plural “amino-group-protecting groups”, “carboxyl-group-protecting groups” and/or “hydroxyl-group-protecting groups”, they may be the same or different and are appropriately selected according to the moieties in need of protection.

The reaction to dehydration-condense compound (a-4) and compound (a-2) by amidation is normally conducted in the presence of equivalent amounts of the amino compound and the carboxylic acid, using 1.1 equivalents or so of a condensing agent such as N-ethyl-N′-dimethylaminocarbodiimide or N-hydroxy-benzotriazol, in a solvent such as DMF or methylene chloride at room temperature for 1 hour to 10 hours or so.

### Process 2: Production Method (2) for the Hydrophilic Spacer Having a Partial Structure Represented by General Formula (Ia)

(m=2, m′=0, m″=2)

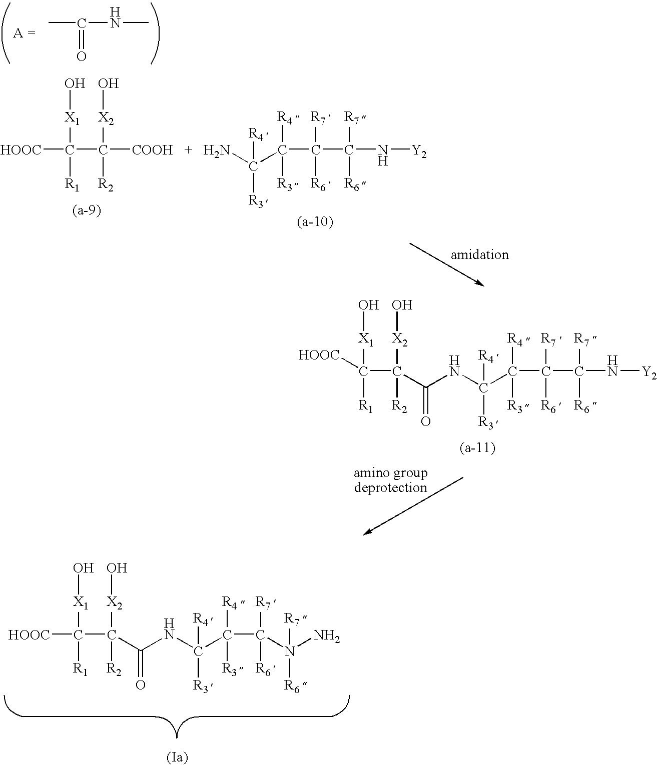

In the formulas, Y₂ is an amino-group-protecting group. R_{3′}, has the same definition as R₃ and R_{3″} has the same definition as R₃. R_{4′} has the same definition as R₄ and R_{4″} has the same definition as R₄. R_{6′} has the same definition as R₆ and R_{6″} has the same definition as R₆. R_{7′} has the same definition as R₇ and R_{7″} has the same definition as R₇. The definitions for the other individual symbols are as described above. As examples of the amino-group-protecting group, the same as those described above can be mentioned. Amino group deprotection is appropriately performed using known methods and reagents according to the protective group used.

The reaction to dehydration-condense compound (a-9) and compound (a-10) by amidation is normally conducted in the presence of equivalent amounts of the amino compound and the carboxylic acid, using 1.1 equivalents or so of a condensing agent such as N-ethyl-N′-dimethylaminocarbodiimide or N-hydroxy-benzotriazol, in a solvent such as DMF or methylene chloride at room temperature for 1 hour to 10 hours or so.

### Process 3: Production Method (3) for the Hydrophilic Spacer Having a Partial Structure Represented by General Formula (Ia)

(m=1, m′=0, m″=0)

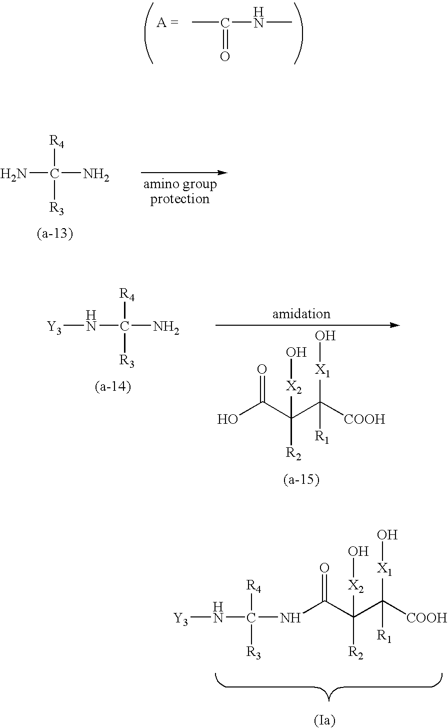

In the formulas, Y₃ is an amino-group-protecting group, and the definitions for the other individual symbols are as described above. As examples of the amino-group-protecting group, the same as those described above can be mentioned.

The reaction to dehydration-condense compound (a-14) and compound (a-15) by amidation is normally conducted in the presence of equivalent amounts of the amino compound and the carboxylic acid, using 1.1 equivalents or so of a condensing agent such as N-ethyl-N′-dimethylaminocarbodiimide or N-hydroxy-benzotriazol, in a solvent such as DMF or methylene chloride at room temperature for 1 hour to 10 hours or so.

### Process 4: Production Method for the Hydrophilic Spacer Having a Partial Structure Represented by General Formula (Ib)

(n−1=n′−1=n₂)

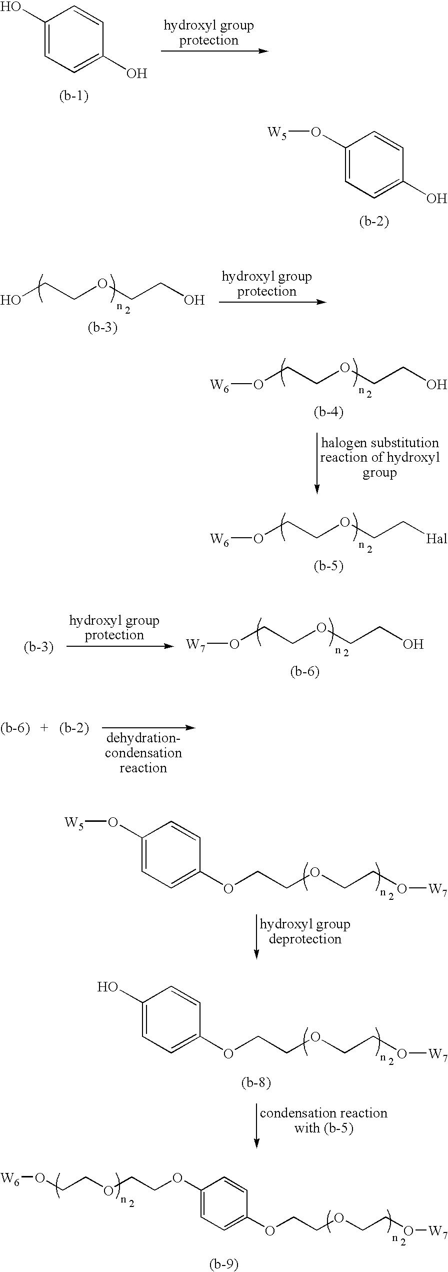

In the formulas, W₅-W₇ are hydroxyl-group-protecting groups, Hal represents a halogen atom (chlorine atom, bromine atom, iodine atom, fluorine atom), and the definitions for the other individual symbols are as described above. As examples of the hydroxyl-group-protecting group, the same as those described above can be mentioned. Note that n₂ is n−1 or n′−1 (n and n′ are as described above).

Hydroxyl group protection and deprotection is appropriately performed using known methods and reagents according to the protective group used.

The halogen substitution reaction of compound (b-4) to compound (b-5) is normally conducted by reacting 2-3 equivalents of carbon tetrabromide and 1-2 equivalents of triphenylphosphine to 1 equivalent of the alcohol compound in a solvent such as methylene chloride, at 0° C. to room temperature, is for 1 hour to several hours.

The dehydration-condensation reaction of compound (b-6) and compound (b-2) is normally conducted by reacting 1 equivalent of the alcohol compound and 1 equivalent of tributylphosphine in a toluene solvent at room temperature for 1 hour or so, adding thereto 1 equivalent of the phenol compound and a condensing agent such as 1,1′-azobis(N,N-dimethylformamide), and allowing the reaction at 0-50° C. for several hours to overnight.

The condensation reaction of compound (b-8) and compound (b-5) is normally conducted by reacting 1 equivalent of the phenol compound and about 10 times equivalents of a strong base like sodium hydride in excess at 0-10° C. in a solvent such as THF for 10-60 minutes or so, adding thereto 2 equivalents or so of the halogen compound, and allowing the reaction at room temperature for 1-10 hours or so.

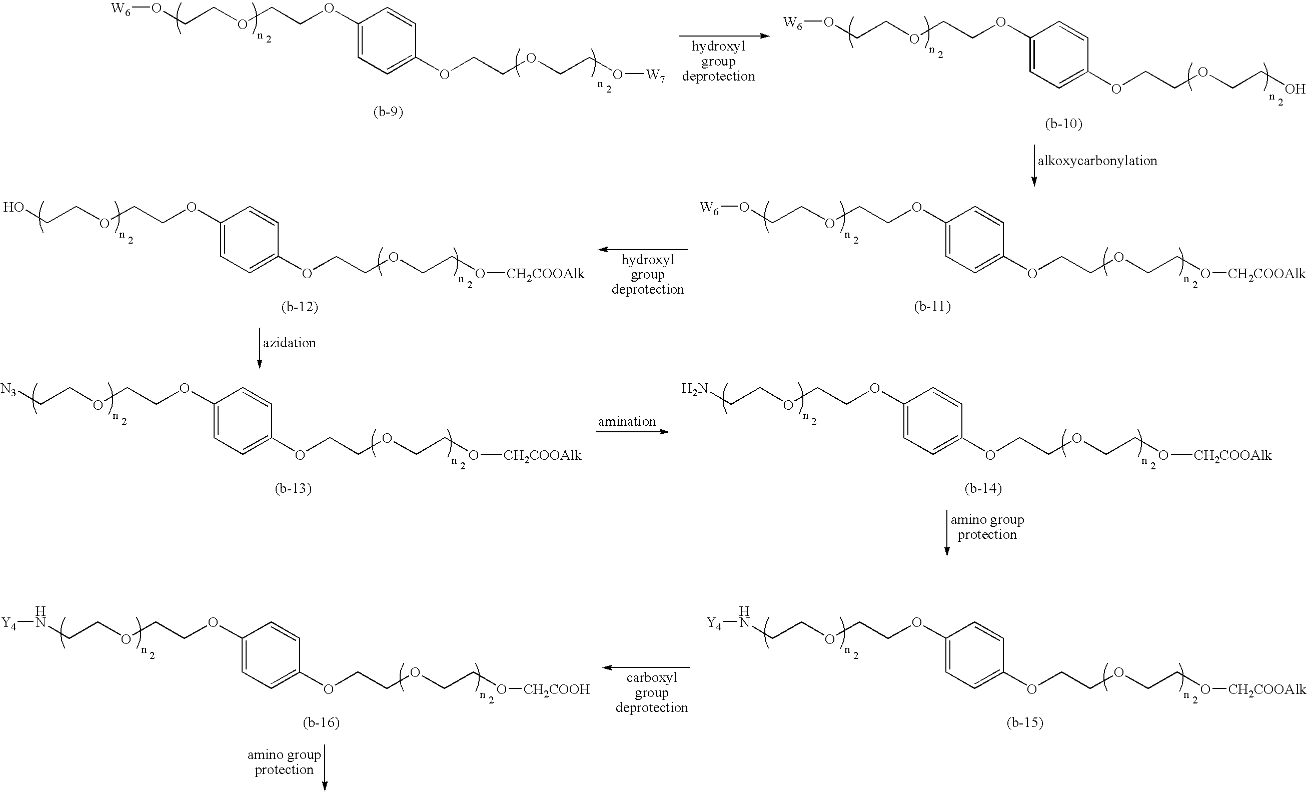

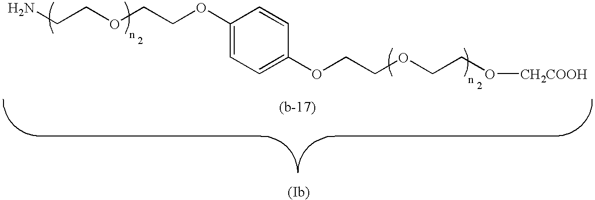

In the formulas, Alk is a linear or branched alkyl group having 1-3 carbon atoms (defined as described above), Y₄ is an amino-group-protecting group, and the definitions for the other individual symbols are as described above. As examples of the hydroxyl-group-protecting group and the amino-group-protecting group, the same as those described above can be mentioned.

Hydroxyl group or amino group deprotection or carboxyl group deprotection is appropriately performed using known methods and reagents according to the protective group used.

Alkoxycarbonylation of compound (b-10) to compound (b-11) is normally conducted by reacting 1 equivalent of the alcohol compound and 3-5 times equivalents or so of a strong base like sodium hydride in excess at 0-10° C. in a solvent such as THF, for 10-60 minutes or so, adding thereto 3-5 times equivalents or so of the halogen compound (bromoacetic acid-tert-butyl ester) in excess, and allowing the reaction at room temperature for 1-10 hours or so.

Azidation of compound (b-12) to compound (b-13) is normally conducted by reacting 1 equivalent of the alcohol compound, 1.5 equivalents or so of p-toluenesulfonyl chloride, and 0.2 equivalents or so of a base like 4-dimethylaminopyridine in a solvent such as pyridine at 30-50° C. for several hours, isolating the O-tosyl compound obtained, adding thereto about 10 times equivalents or so of sodium azide in excess, and allowing the reaction in a solvent such as DMF at 50-90° C. for several hours.

Amination of compound (b-13) to compound (b-14) is normally achieved by reacting 1 equivalent of the azide compound, using 0.1 equivalent or so of a catalyst like palladium hydroxide, in the presence of a solvent such as methanol under 1 to several atmospheric pressures of hydrogen at room temperature for several hours.

### Process 5: Production Method for the Hydrophilic Spacer Having a Partial Structure Represented by General Formula (Ic)

In each structural formula, particular groups and particular compounds are shown in some cases, which, however, are given for exemplification and are not to be construed as limiting. They are appropriately variable, as long as they retain an equivalent function.

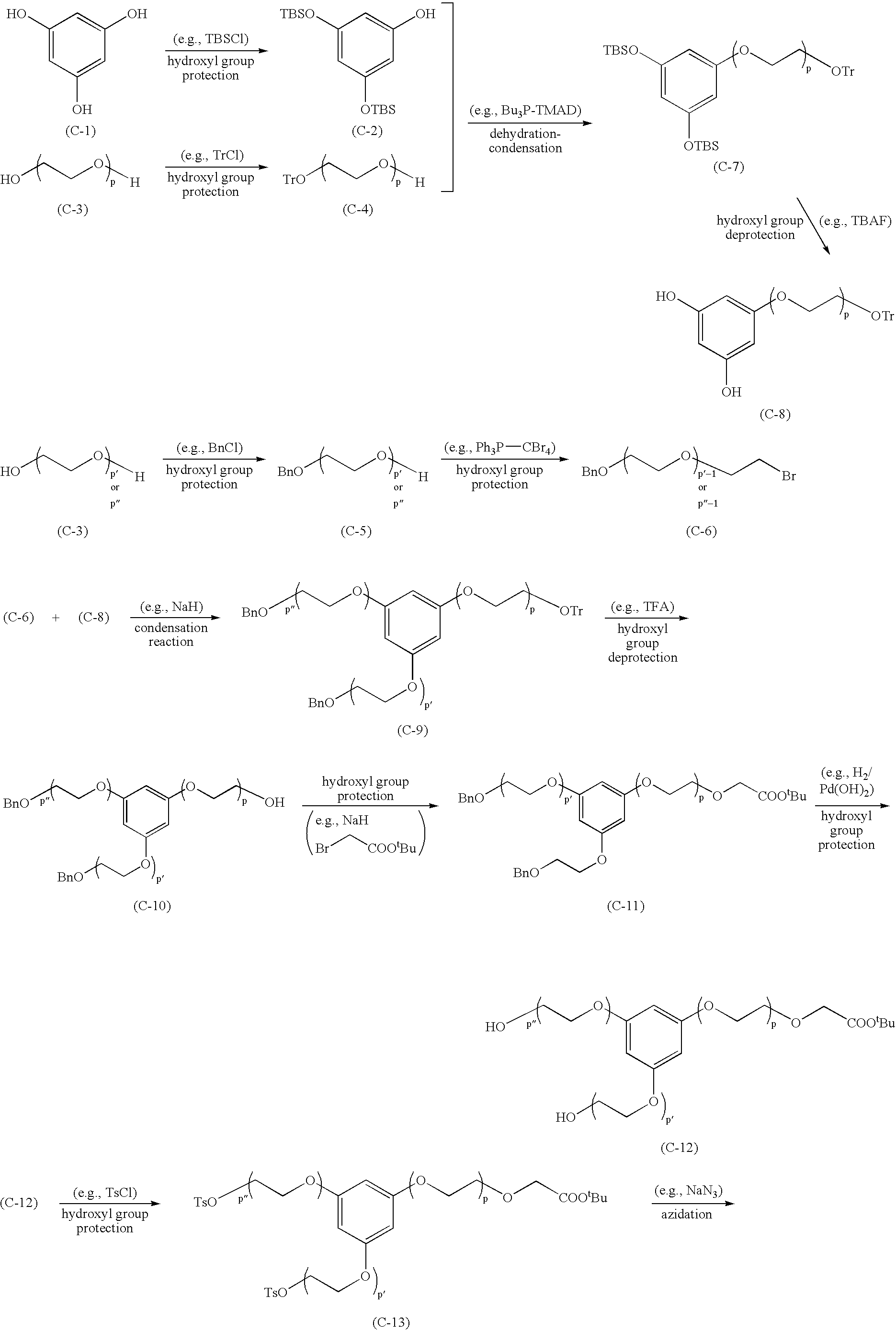

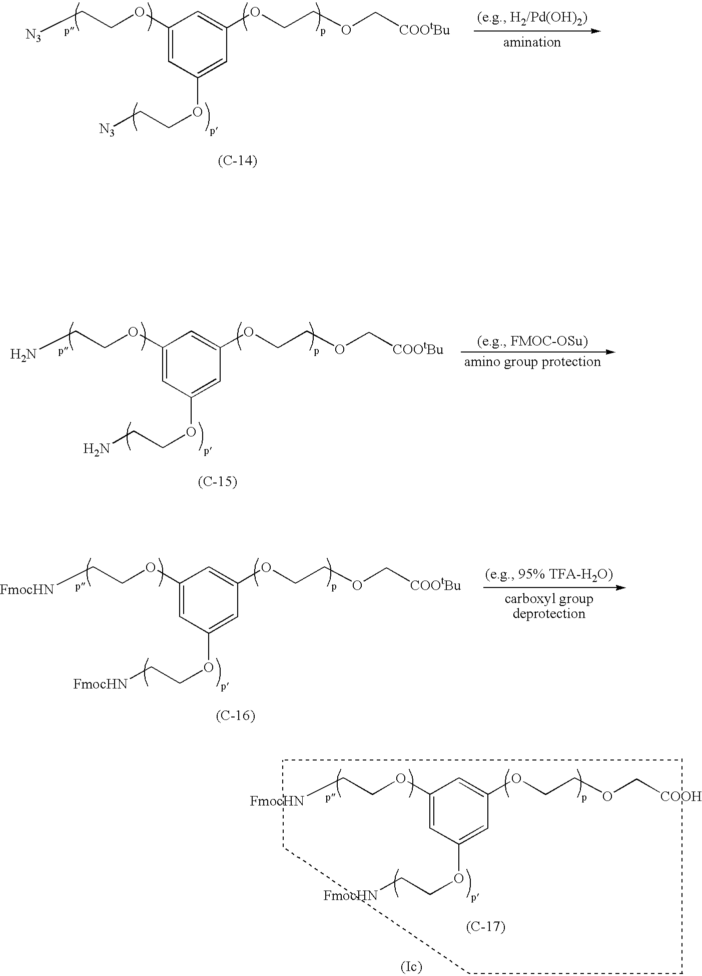

In the formulas, the definitions for individual symbols are as described above. The hydroxyl-group-protecting groups, amino-group-protecting groups and carboxyl-group-protecting groups in the formulas are given for exemplification, in addition to which groups optionally chosen groups in common use in the art are used. Specifically, the same as those described above can be mentioned as examples. It will be obvious to those skilled in the art that amino group protection, carboxyl group deprotection, and hydroxyl group protection and deprotection can be appropriately performed using known methods and reagents according to the protective group used, in addition to those described in the present specification.

Hydroxyl group protection of compound (c-1) to compound (c-2), when using TBS, for example, as the protective group, is normally conducted by reacting 1 equivalent of the phenol compound, 3 equivalents or so of a base (e.g., imidazole) and 2 equivalents or so of silyl chloride in a solvent such as DMF at room temperature for 10 hours or so.

The dehydration-condensation reaction of compound (c-2) and compound (c-4) is normally conducted by reacting 1 equivalent of the alcohol compound and 1 equivalent of tributylphosphine in a toluene solvent at room temperature for 1 hour or so, adding thereto 1.3 equivalents of the phenol compound and 1.3 equivalents of a condensing agent such as 1,1′-azobis(N,N-dimethylformamide), and allowing the reaction at room temperature for several hours to overnight.

Hydroxyl group deprotection of compound (c-7) to compound (c-8) is normally conducted by reacting 1 equivalent of the phenol-protected compound (e.g., silyl-protected compound) and 1.2 equivalents or so of tetrabutylammonium fluoride in a solvent such as THF at room temperature for 1 hour or so.

The condensation reaction of compound (c-8) and compound (c-6) is normally conducted by reacting 1 equivalent of the phenol compound and about 5.2 equivalents of a strong base like sodium hydride in excess at room temperature in a solvent such as THF or DMF for 10-60 minutes or so, adding thereto 4 equivalents or so of a halide (e.g., alkyl bromide), and allowing the reaction at room temperature for about 4 hours or so. By this condensation reaction, compound (c-9) is obtained.

Hydroxyl group deprotection of compound (c-9) to compound (c-10) is normally conducted by reacting 1 equivalent of the phenol-protected compound (e.g., trityl-protected compound) in a solvent such as methylene chloride that contains TEA at room temperature for about 1 hour or so.

Hydroxyl group protection of compound (c-10) to compound (c-11), when using a tert-butoxycarbonyl group, for example, as the protective group, is normally conducted by reacting 1 equivalent of the alcohol compound, about 4 equivalents of a strong base such as sodium hydride, and about 4 equivalents of bromoacetic acid tert-butyl ester in a solvent such as THF or DMF at room temperature for about 4 hours or so.

Hydroxyl group deprotection of compound (c-11) to compound (c-12) is normally conducted by reacting 1 equivalent of a phenol-protected compound (e.g., benzyl-protected compound) and a catalytic amount of palladium hydroxide in a hydrogen gas atmosphere in a solvent such as methanol at room temperature for about 6 hours or so.

Hydroxyl group protection of compound (c-12) to compound (c-13), when using Ts, for example, as the protective group, is normally conducted by reacting 1 equivalent of the alcohol compound, a catalytic amount of a base such as DMAP, and about 6 equivalents of tosyl chloride in a solvent such as pyridine at room temperature to 40° C. for about 2 hours or so.

Azidation of compound (c-13) to compound (c-14) is conducted by reacting 1 equivalent of the tosyl compound and about 15 equivalents of sodium azide in a solvent such as DMF at about 60° C. for about 2 hours or so.

Amination of compound (c-14) to compound (c-15) and amino-group-protecting group introduction to compound (C-16) are normally conducted by reacting 1 equivalent of the phenol-protected compound (benzyl-protected compound) and a catalytic amount of palladium hydroxide in a hydrogen gas atmosphere in a solvent such as methanol at room temperature for about 1 hour or so, adding to the amine compound obtained (c-15) about 0.84 equivalents of 9-fluorenylmethylsuccinimidyl carbonate and about 1.5 equivalents of a base like triethylamine, and allowing the reaction in a solvent such as THF at room temperature for about 1 hour or so.

Carboxyl group deprotection of compound (c-16) to compound (c-17) is normally conducted by reacting 1 equivalent of the phenol-protected compound (e.g., t-butyl-protected compound) in an aqueous solution that contains TFA at room temperature for about 10 hours or so.

### Process 6: Production Method for the Hydrophilic Spacer Having a Partial Structure Represented by General Formula (Id)

### (R₁₀=R₉=Hydrogen Atom, R₈=Hydrogen Atom, X₄=Single Bond)

In each structural formula, particular groups and particular compounds are shown in some cases, which, however, are given for exemplification and are not to be construed as limiting. They are appropriately variable, as long as they retain an equivalent function.

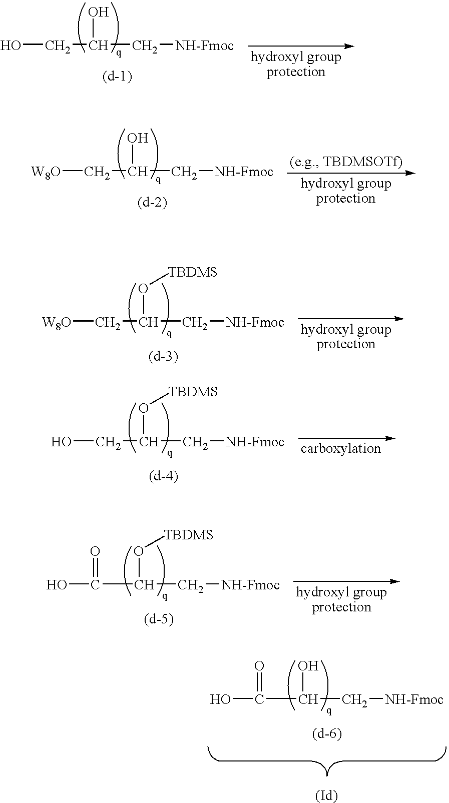

In the formulas, W₈ is a hydroxyl-group-protecting group, and the definitions for the other symbols are as described above. As examples of the hydroxyl-group-protecting group, the same as those described above can be mentioned. Hydroxyl group deprotection is appropriately performed using known methods and reagents according to the protective group used.

Carboxylation from compound (d-4) to compound (d-5) is normally achieved by reacting 1 equivalent of the alcohol compound with 10 equivalents of sodium periodate, 0.4 equivalents or so of an oxidant like ruthenium chloride hydrate (III) in the presence of a solvent such as water, acetonitrile or dichloromethane at room temperature for several hours.

### Process 7: Production Method (1) for the Hydrophilic Spacer Having a Partial Structure Represented by General Formula (Ie)

In each structural formula, particular groups and particular compounds are shown in some cases, which, however, are given for exemplification and are not to be construed as limiting. They are appropriately variable, as long as they retain an equivalent function.

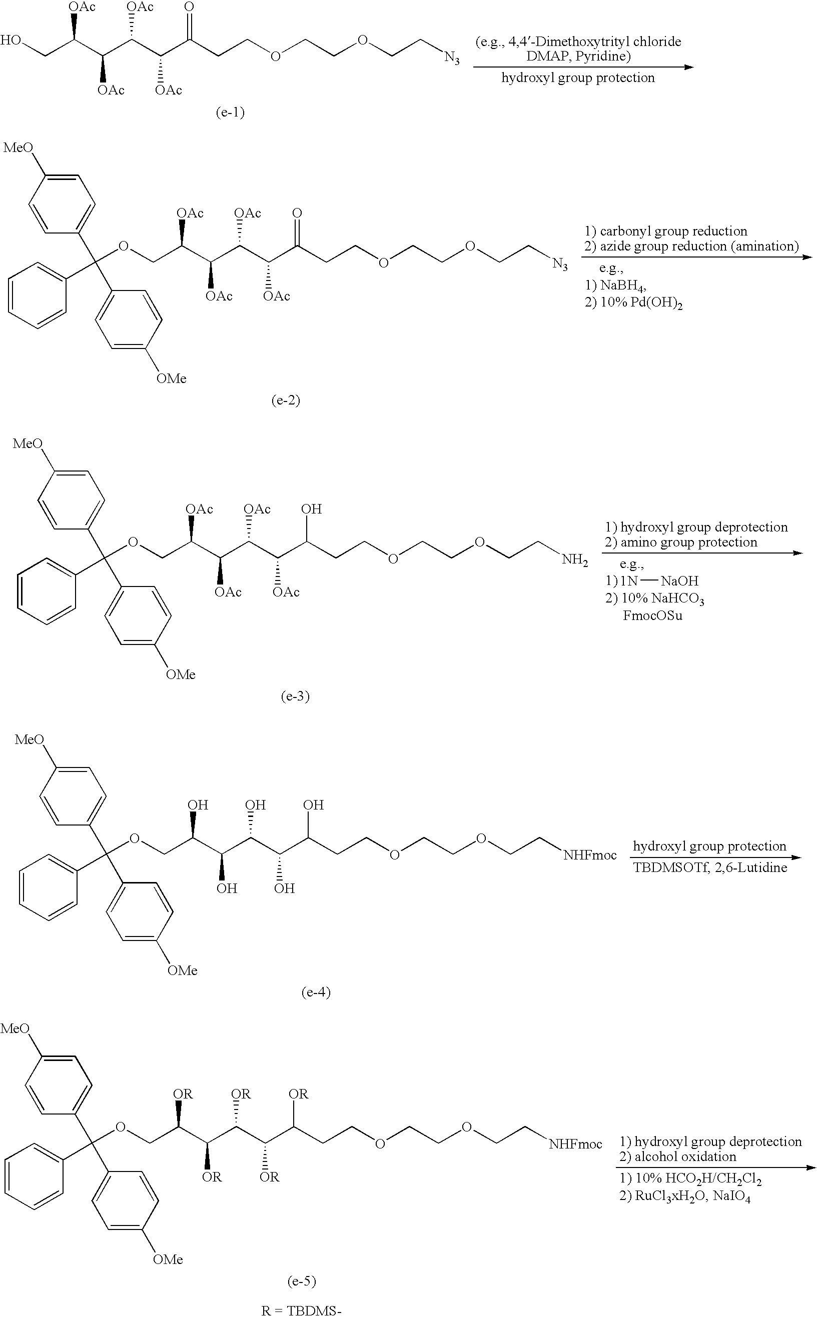

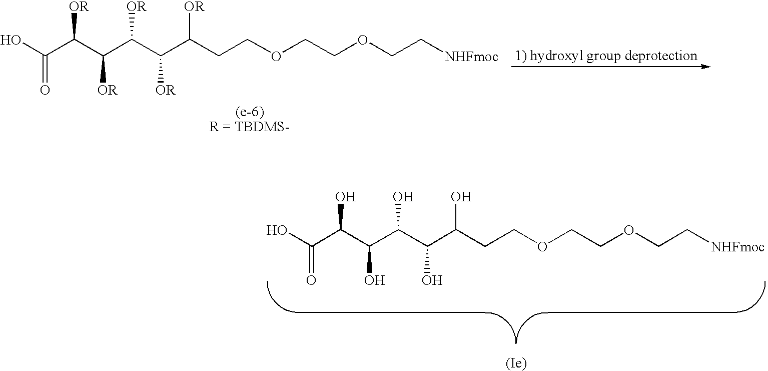

As examples of the hydroxyl-group-protecting group and the amino-group-protecting group, the same as those described above can be mentioned. Hydroxyl group deprotection is appropriately performed using known methods and reagents according to the protective group used.

The carbonyl group reduction reaction of compound (e-2) to compound (e-3) is conducted by reacting 1.2 equivalents or so of a reducing agent like NaBH₄ in a solvent such as methanol, and subsequently normally carrying out an azide group reduction reaction Lamination) of 1 equivalent of the azide compound and 0.1 equivalent or so of a catalyst like palladium hydroxide in the presence of a solvent such as methanol under 1 to several atmospheric pressures of hydrogen at room temperature for several hours.

Hydroxyl group deprotection of compound (e-3) to compound (e-4) can be conducted by reacting an alkali such as 1N sodium hydroxide in a mixed solvent of dioxane, water and the like, and subsequently protecting the amino group in the same manner as the reaction from (c-15) to (c-16).

Hydroxyl group protection of compound (e-4) to compound (e-5) can, for example, be conducted by reacting 20 equivalents or so of TBDMS-OTf in the presence of 2,6-Lutidine and the like.

Hydroxyl group deprotection of compound (e-5) to compound (e-6) can be conducted by allowing the reaction with 10% formic acid/dichloromethane, and subsequently oxidizing the alcohol in the same manner as the reaction from compound (d-4) to compound (d-5).

Process 8: Production Method (2) for the hydrophilic spacer having a partial structure represented General Formula (Ie)
(R₁₃-R₁₆=H, R₁₁=H, R₁₂=H, r=1)

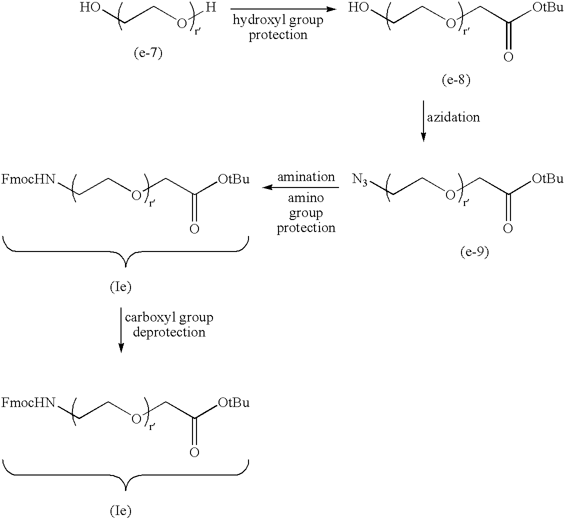

In the formulas, the definitions for individual symbols are as described above. The hydroxyl-group-protecting groups, amino-group-protecting groups and carboxyl-group-protecting groups in the formulas are given for exemplification, in addition to which groups optionally chosen groups in common use in the art are used. Specifically, the same as those described above can be mentioned as examples. It will be obvious to those skilled in the art that amino group protection, carboxyl group deprotection and hydroxyl group protection can be appropriately performed using known methods and reagents according to the protective group used, in addition to those described in the present specification.

Azidation from compound (e-8) to compound (e-9) is conducted by reacting 1 equivalent of compound (e-8), a catalytic amount of a base such as DMAP, and about 10 equivalents of tosyl chloride in a solvent such as methylene chloride at room temperature to 40° C. for about 2 hours to overnight to yield a tosyl derivative of compound (e-8), and reacting 1 equivalent of the tosyl derivative obtained with about 15 equivalents of sodium azide in a solvent such as DMSO at about 60-70° C. for about 5 hours or so.

By aminating compound (e-9), and subsequently protecting the amino group, a compound having a partial structure represented by Formula (Ie) is obtained.

Usually, the amine compound is obtained by reacting 1 equivalent of compound (e-9) and a catalytic amount of palladium hydroxide in a hydrogen atmosphere in a solvent such as methanol or ethanol at room temperature for about 1-2 hours or so. Next, an amino-group-protecting group is introduced by reacting the amine compound obtained in accordance with a conventional method using, for example, 9-fluorenylmethylsuccinimidyl carbonate and the like, in the presence of a base like triethylamine in a solvent such as THF.

In the present invention, in addition to the compounds mentioned above as hydrophilic spacers, the polymers obtained by polymerizing them can also be used as hydrophilic spacers. For the polymerization, various methods in general use in the art can be employed.

Specifically, the polymerization is conducted by subjecting compounds described above to chemical reactions such as amidation, N-substitutional amidation, Schiff base formation (after Schiff base formation, the relevant portion may be subjected to a reduction reaction), esterification, and epoxy cleavage reaction with an amine or a hydroxyl group. Although the polymerization reaction can be conducted while the starting monomer component is in a free state, it is preferable, because of the ease of the subsequent purification step, to immobilize the starting monomer component onto a metal surface and then conduct the polymerization reaction on the metal surface. The reagents and reaction conditions used for these reactions are according to methods in common use in the art.

### EXAMPLES

The present invention is hereinafter described in more detail by means of the following production examples, an example and an experimental example, which examples, however, are not to be construed as limiting the scope of the present invention.

### Production Example 1

### Synthesis of 17-allyl-14-(tert-butyl-dimethyl-silanyloxy)-1-hydroxy-12-{2-[4-(7-(tert-butyl-dimethyl-silanyloxy-carbonyl)heptanoyl-oxy)-3-methoxy-cyclohexyl]-1-methyl-vinyl}-23,25-dimethoxy-13,19,21,27-tetramethyl-11,28-dioxa-4-aza-tricyclo[22.3.1.0^{4,9}]octacos-18-ene-2,3,10,16-tetraone

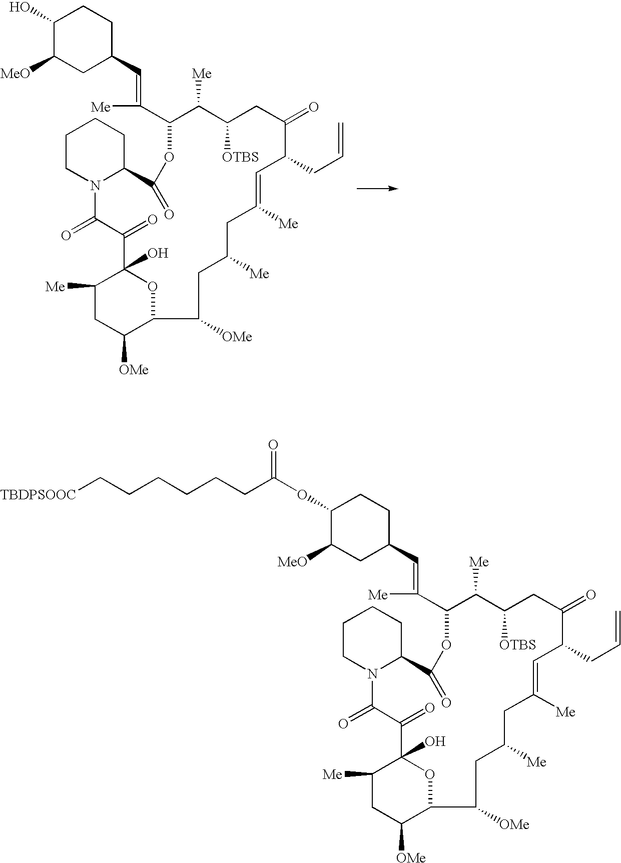

A mixture of 17-allyl-14-(tert-butyl-dimethyl-silanyloxy)-1-hydroxy-12-[2-(4-hydroxy-3-methoxy-cyclohexyl)-1-methyl-vinyl]-23,25-dimethoxy-13,19,21,27-tetramethyl-11,28-dioxa-4-aza-tricyclo[22.3.1.0^{4,9}]octacos-18-ene-2,3,10,16-tetraone (FK506; 138 mg, 0.15 mmol), O-mono(tert-butyl-dimethyl-silanyl)octanedioic acid (86.7 mg, 0.218 mmol), dimethylaminopyridine (DMAP; 16.5 mg, 0.098 mmol), 1-[3-(dimethylamino)propyl]-3-ethylcarbodiimide hydrochloride (EDC.HCl; 69.1 mg, 0.261 mmol) and methylene chloride (CH₂Cl₂; 1 ml) was stirred at room temperature for 1.5 hours. The reaction product was poured over an ethyl acetate-water mixed fluid and extracted. The organic phase obtained was washed with water and brine, after which it was dried with magnesium sulfate (MgSO₄). After the MgSO₄ was separated by filtration, concentration under reduced pressure was conducted. The residue thus obtained was purified using a silica gel column (eluted with 20% AcOEt (in n-hexane)) to yield the desired 17-allyl-14-(tert-butyl-dimethyl-silanyloxy)-1-hydroxy-12-{2-[4-(7-(tert-butyl-dimethyl-silanyloxy-carbonyl)heptanoyl-oxy)-3-methoxy-cyclohexyl]-1-methyl-vinyl}-23,25-dimethoxy-13,19,21,27-tetramethyl-11,28-dioxa-4-aza-tricyclo[22.3.1.0^{4,9}]octacos-18-ene-2,3,10,16-tetraone (44 mg, 24.6%).

¹H-NMR (CDCl₃) δ: −0.1-0.1 (12H, m), 0.7-2.6 (47H, m), 0.85 and 0.86 (18H, s), 1.50 (3 H, s), 1.63 (3H, s), 2.75 (1H, m), 3.31 (3H, s), 3.35 (3H, s), 3.39 (3H, s), 4.05 (1H, m), 3.0-4.4 (6H), 4.5-5.8 (9H, m).

### Production Example 2

### Synthesis of 17-allyl-1,14-dihydroxy-12-{2-[4-(7-carboxy-heptanoyl-oxy)-3-methoxy-cyclohexyl]-1-methyl-vinyl}-23,25-dimethoxy-13,19,21,27-tetramethyl-11,28-dioxa-4-aza-tricyclo[22.3.1.0^{4,9}]octacos-18-ene-2,3,10,16-tetraone

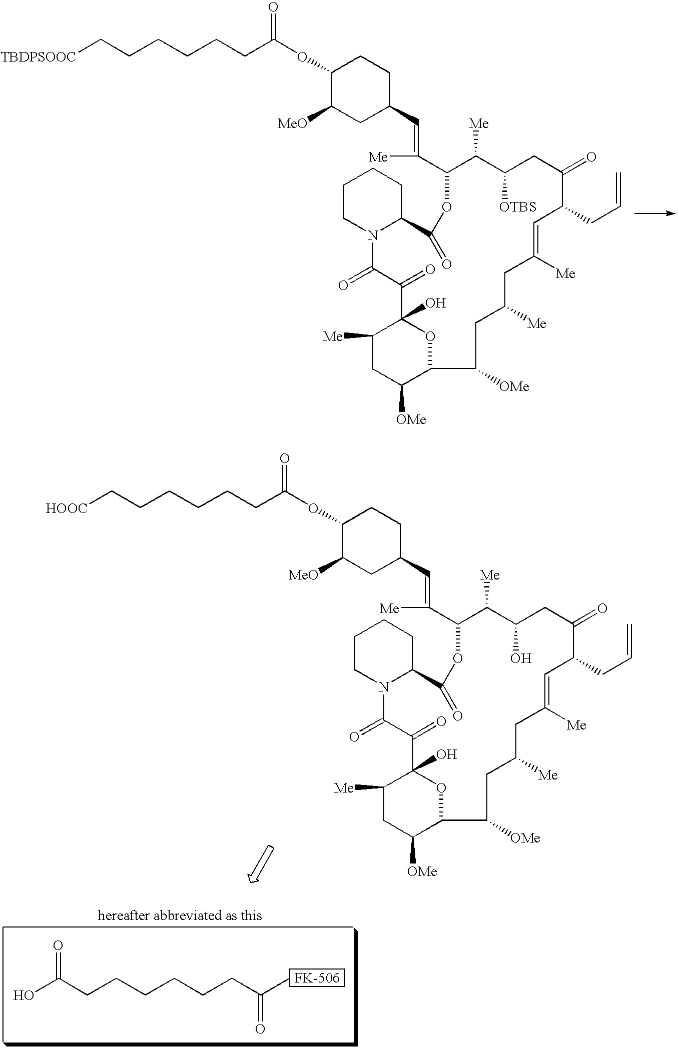

To a mixture of the 17-allyl-14-(tert-butyl-dimethyl-silanyloxy)-1-hydroxy-12-{2-[4-(7-(tert-butyl-dimethyl-silanyloxy-carbonyl)heptanoyl-oxy)-3-methoxy-cyclohexyl]-1-methyl-vinyl}-23,25-dimethoxy-13,19,21,27-tetramethyl-11,28-dioxa-4-aza-tricyclo[22.3.1.0^{4,9}]octacos-18-ene-2,3,10,16-tetraone prepared in Production Example 1 (44 mg, 0.037 mmol) and acetonitrile (0.88 ml), 46-48% aqueous hydrogen fluoride (HF) (0.12 ml) was gently added, and this was followed by overnight stirring at room temperature. The reaction product was poured over an ethyl acetate-water mixed fluid and extracted. The organic phase obtained was washed with water and brine, after which it was dried with magnesium sulfate (MgSO₄). After the MgSO₄ was separated by filtration, concentration under reduced pressure was conducted. The residue thus obtained was purified using a silica gel column (5% methanol (in chloroform)) to yield the desired 17-allyl-1,14-dihydroxy-12-{2-[4-(7-carboxy-heptanoyl-oxy)-3-methoxy-cyclohexyl]-1-methyl-vinyl}-23,25-dimethoxy-13,19,21,27-tetramethyl-11,28-dioxa-4-aza-tricyclo[22.3.1.0^{4,9}]octacos-18-ene-2,3,10,16-tetraone (14.2 mg, 40%).

¹H-NMR (CDCl₃) δ: 0.7-2.6 (47H, m), 1.50 (3H, s), 1.63 (3H, s), 2.75 (1H, m), 3.31 (3H, s), 3.35 (3H, s), 3.39 (3H, s), 4.05 (1H, m), 3.0-4.4 (6H), 4.5-5.8 (11H, m).

MS (m/z): 960 (M⁺)

### Production Example 3

### Synthesis (1-1) of Hydrophilic Spacer Molecule

### Synthesis of 2-(2-{2-[2-(2-trityloxy-ethoxy)-ethoxy]-ethoxy}-ethoxy)ethanol

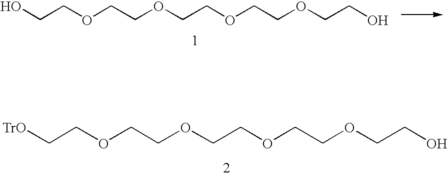

Pentaethylene glycol (compound 1; 10 g, 42.0 mmol) was dissolved in pyridine (100 ml), triphenylmethyl chloride (11.6 g, 41.6 mmol) and 4-dimethylaminopyridine (0.9 g, 7.4 mmol) were added at room temperature, and this was followed by overnight stirring at 35° C. This was concentrated under reduced pressure; the residue obtained was dissolved in chloroform, the organic phase was washed with saturated aqueous sodium hydrogen carbonate and saturated brine, after which it was dried with sodium sulfate. The solid was removed by cotton filtration and washed with chloroform, and the filtrate and the washings were combined and concentrated under reduced pressure. The residue obtained was subjected to silica gel column chromatography (Kanto Chemical 60N; 600 ml) with an eluent (60:1 chloroform (CHCl₃)-methanol (MeOH)) to yield the desired 2-(2-{2-[2-(2-trityloxy-ethoxy)-ethoxy]-ethoxy}-ethoxy)ethanol (compound 2; 10.4 g, 51.2%).

¹H-NMR (CDCl₃) δ: 2.53 (1H, t), 3.16 (2H, t), 3.49-3.63 (18H, m), 7.14-7.41 (15H, m).

### Production Example 4

### Synthesis (1-2) of Hydrophilic Spacer Molecule

### Synthesis of [2-(2-{2-[2-(2-trityloxy-ethoxy)-ethoxy]-ethoxy}-ethoxy)-ethoxy]acetic acid

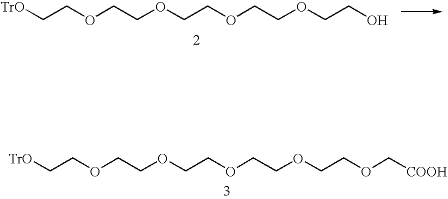

The compound 2 obtained in Production Example 3 (10.2 g, 21.2 mmol) was dissolved in a mixed solvent of tetrahydrofuran (THF; 200 ml) and DMF (50 ml), sodium hydride (3.1 g; oily, 60 wt %) was added little by little at 0° C., and this was followed by stirring at room temperature for 30 minutes. After this was cooled to 0° C., bromoacetic acid (6.5 g, 46.8 mmol) was added little by little, and this was followed by stirring at room temperature for 30 minutes. Subsequently, sodium hydride (11.6 g; oily, 60 wt %) was further added little by little at room temperature, and this was followed by stirring at room temperature for 1 hour. The reaction solution was cooled to 0° C., and water (25 ml) was gradually added, after which the reaction solution was concentrated under reduced pressure until the volume thereof became about 100 ml. Ethyl acetate (200 ml) and brine (100 ml) were added thereto, and 2M aqueous potassium hydrogen sulfate was added with stirring to obtain a pH of 6. The organic phase was extracted and concentrated under reduced pressure at 30° C.; the residue obtained was subjected to silica gel column chromatography (Kanto Chemical 60N; 400 ml) with an eluent (85:15 CHCl₃-MeOH) to yield a crude product of the desired [2-(2-{2-[2-(2-trityloxy-ethoxy)-ethoxy]-ethoxy}-ethoxy)-ethoxy]acetic acid (compound 3) (12.4 g).

¹H-NMR (CDCl₃) δ: 3.34 (2H, t), 3.76-3.84 (20H, m), 4.13 (2H, s), 7.30-7.83 (15H, m).

### Production Example 5

### Synthesis (1-3) of Hydrophilic Spacer Molecule

### Synthesis of [2-(2-{2-[2-(2-trityloxy-ethoxy)-ethoxy]-ethoxy}-ethoxy)-ethoxy]acetic acid benzyl ester

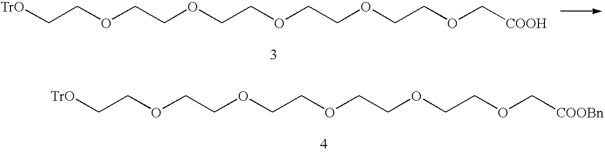

The crude product of compound 3 obtained in Production Example 4 (12.4 g) was dissolved in methylene chloride (100 ml), and 4-dimethylaminopyridine (0.29 g, 2.4 mmol) and benzyl alcohol (3.1 ml, 30.0 mmol) were added. This was cooled to 0° C., water-soluble carbodiimide (N-ethyl-N′-(3′-dimethylaminopropyl)carbodiimide; WSC; 4.5 g, 23.5 mmol) was added, and this was followed by overnight stirring at room temperature. The reaction solution was extracted with chloroform, and the organic phase was washed with saturated aqueous sodium hydrogen carbonate and saturated brine, after which it was dried with sodium sulfate. The solid was removed by cotton filtration and washed with chloroform, and the filtrate and the washings were combined and concentrated under reduced pressure. The residue obtained was subjected to silica gel column chromatography (Kanto Chemical 60N; 600 ml) with an eluent (1:1 ethyl acetate-hexane) to yield the desired [2-(2-{2-[2-(2-trityloxy-ethoxy)-ethoxy]-ethoxy}-ethoxy -ethoxy]acetic acid benzyl ester (compound 4; 12.0 g, 90.1%, 2 steps).

¹H-NMR (CDCl₃) δ: 3.16 (2H, t), 3.55-3.65 (20H, m), 4.11 (2H, s), 5.11 (2H, s), 7.15-7.40 (20H, m).

### Production Example 6

### Synthesis (1-4) of Hydrophilic Spacer Molecule

### Synthesis of [2-(2-{2-[2-(2-hydroxy-ethoxy)-ethoxy]-ethoxy}-ethoxy)-ethoxy]acetic acid benzyl ester

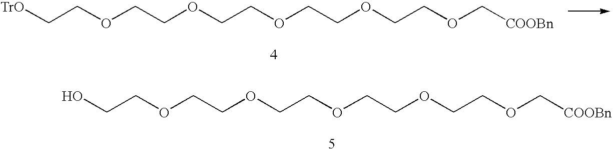

The compound 4 obtained in Production Example 5 (12.0 g) was dissolved in a 5% solution of trifluoroacetic acid in methylene chloride (150 ml), water (10 ml) was added at 0° C., and this was followed by stirring at 0° C. for 20 minutes. The reaction solution was poured over saturated aqueous sodium hydrogen carbonate, extracted, and dried with sodium sulfate. The solid was removed by cotton filtration and washed with chloroform, and the filtrate and the washings were combined and concentrated under reduced pressure. The residue obtained was subjected to silica gel column chromatography (Kanto Chemical 60N; 400 ml) with an eluent (1000:15 CHCl₃-MeOH) to yield the desired [2-(2-{2-[2-(2-hydroxy-ethoxy)-ethoxy]-ethoxy}-ethoxy)-ethoxy]acetic acid benzyl ester (compound 5; 7.0 g, 95%).

¹H-NMR (CDCl₃) δ: 2.80 (1H, t), 3.62-3.76 (20H, m), 4.22 (2H, s), 5.20 (2H, s), 7.36-7.41 (5H, m).

### Production Example 7

### Synthesis (1-5) of Hydrophilic Spacer Molecule

### Synthesis of [2-(2-{2-[2-(2-azido-ethoxy)-ethoxy]-ethoxy}-ethoxy)-ethoxy]acetic acid benzyl ester

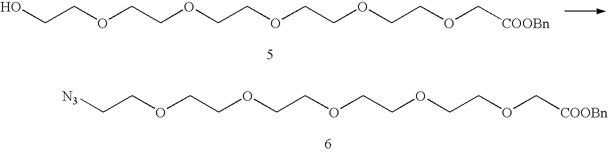

The compound 5 obtained in Production Example 6 (7.0 g, 18.1 mmol) and 4-dimethylaminopyridine (0.4 g, 3.3 mmol) were dissolved in pyridine (45 ml), and this solution was cooled to 0° C. p-Toluenesulfonyl chloride (5.2 g, 27.2 mmol) was added thereto, this was followed by overnight stirring at room temperature, p-toluenesulfonyl chloride (3.1 g, 16.2 mmol) and 4-dimethylaminopyridine (120 mg, 0.98 mmol) were further added, and this was followed by stirring at 30° C. for 2 hours. The reaction solution was cooled to 0° C., water (3 ml) was added, concentration under reduced pressure was conducted, the residue obtained was dissolved in ethyl acetate, and the organic phase was washed with saturated aqueous sodium hydrogen carbonate and saturated brine, after which it was dried with sodium sulfate. The solid was removed by cotton filtration and washed with ethyl acetate, and the filtrate and the washings were combined and concentrated under reduced pressure. The residue obtained was dissolved in DMF (50 ml), sodium azide (11.8 g, 0.18 mol) was added, and this was followed by stirring at 60° C. for 1 hour. The reaction solution was extracted with ethyl acetate, and the organic phase was washed with saturated aqueous sodium hydrogen carbonate and saturated brine, after which it was dried with sodium sulfate. The solid was removed by cotton filtration and washed with ethyl acetate, and the filtrate and the washings were combined and concentrated under reduced pressure. The residue obtained was subjected to silica gel column chromatography (Kanto Chemical 60N; 250 ml) with an eluent (3:1 ethyl acetate-hexane) to yield the desired [2-(2-{2-[2-(2-azido-ethoxy)-ethoxy]-ethoxy}-ethoxy)-ethoxy]acetic acid benzyl ester (compound 6; 3.3 g, 44.3%).

¹H-NMR (CDCl₃) δ: 3.31 (2H, t), 3.54-3.87 (20H, m), 4.13 (2H, s), 5.12 (2H, s), 7.20-7.30 (5H, m).

### Production Example 8

### Synthesis (1-6) of Hydrophilic Spacer Molecule

### Synthesis of [2-(2-{2-[2-(2-amino-ethoxy)-ethoxy]-ethoxy}-ethoxy)-ethoxy]acetic acid

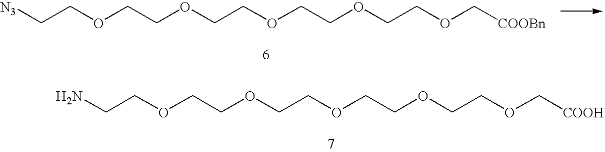

The compound 6 obtained in Production Example 7 (1.94 g, 4.72 mmol) was dissolved in methanol (50 ml), 10% Pd—C (500 mg) was added, and catalytic hydrogenation was conducted at room temperature for 2.5 hours. The solid was removed by Celite filtration and washed with methanol, and the filtrate and the washings were combined and concentrated under reduced pressure to yield the desired [2-(2-{2-[2-(2-amino-ethoxy)-ethoxy]-ethoxy}-ethoxy)-ethoxy]acetic acid (compound 7; 1.4 g, quantitative).

MS (m/z): 296 (M⁺)

### Production Example 9

### Synthesis (1-7) of Hydrophilic Spacer Molecule

### Synthesis of {2-[2-(2-{2-[2-(9H-fluoren-9-yl-methoxycarbonylamino)-ethoxy]-ethoxy}-ethoxy)-ethoxy]-ethoxy}acetic acid

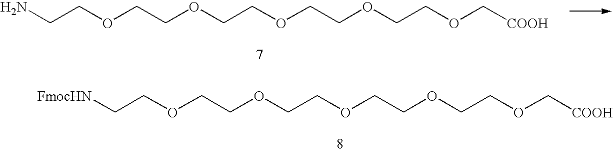

The compound 7 obtained in Production Example 8 (1.25 g, 4.23 mmol) was dissolved in 10% aqueous sodium carbonate (14 ml), 9-fluorenylmethylsuccinimidyl carbonate (2.15 g, 6.37 mmol) in suspension in dimethoxyethane (14 ml) was added drop by drop at room temperature, and this was followed by overnight stirring at room temperature. The solid was separated by filtration with Celite, after which it was washed with chloroform. The filtrate and the washings were combined and extracted with chloroform, and the organic phase was washed with 2M aqueous sodium hydrogen sulfate and saturated brine and dried with sodium sulfate. The solid was removed by cotton filtration and washed with chloroform, and the filtrate and the washings were combined and concentrated under reduced pressure. The residue obtained was subjected to silica gel column chromatography (Kanto Chemical 60N; 150 ml) with an eluent (1000:7 CHCl₃-MeOH) to yield the desired {2-[2-(2-{2-[2-(9H-fluoren-9-yl-methoxycarbonylamino)-ethoxy]-ethoxy}-ethoxy)-ethoxy]-ethoxy}acetic acid (compound 8; 1.38 g, 63.0%).

¹H-NMR (CDCl₃) δ: 3.34 (2H, t), 3.50-3.71 (18H, m), 4.05 (2H, s), 4.12 (1H, t), 4.33 (2H, d), 5.57 (1H, s), 7.22-7.95 (8H, m).

### Production Example 10

### Synthesis of Gold Film Bearing Hydrophilic Spacer: Gold Film Bearing Hexaethylene Glycol Derivative (Kojundo Chemical Laboratory Co., Ltd.; Pure Gold, Purity 99.9% up, Shape 10 mm×10 mm×0.01 mm (Thickness))

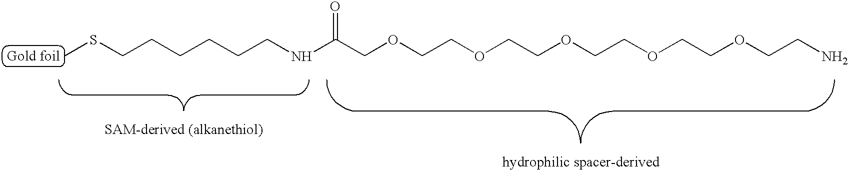

A gold film (about 1 cm²) was immersed in a Piranha solution (30% hydrogen peroxide:con. sulfuric acid=1:4 mixed solution) for several hours and washed with milli Q water (water filtered by pure water production apparatus of Millipore) and ethanol. This was immersed overnight in a 1.5 mM ethanol solution (0.5 ml) of (6-mercapto-hexyl)-carbamic acid 9H-fluoren-9-yl-methyl ester to form SAM on the gold thin film. After completion of the reaction, the gold film was sufficiently washed with ethanol and acetonitrile, and the presence of about 250 Pmol of amine on the gold film was confirmed by the method described in Production Example 12.

The gold film was sufficiently washed with acetonitrile, {2-[2-(2-{2-[2-(9H-fluoren-9-yl-methoxycarbonylamino)-ethoxy]-ethoxy}-ethoxy)-ethoxy]-ethoxy}acetic acid (compound 8 obtained in Production Example 9; 12.5 mg, 0.024 mmol) dissolved in acetonitrile (0.25 ml) was added, benzotriazol-1-yl-oxy-tris-pyrrolidino-phosphonium hexafluorophosphate (PyBOP; 13 mg, 0.025 mmol) dissolved in acetonitrile (0.25 ml), and N,N-diisopropylethylamine (8.9 μl, 0.50 mmol) were further added, and the mixture was shaken overnight at room temperature. The reaction mixture was removed, and the gold film was washed with acetonitrile, and allowed to react overnight under the same conditions. After completion of the reaction, the gold film was sufficiently washed with acetonitrile, acetic acid (0.3 μl, 0.005 mmol) dissolved in acetonitrile (0.25 ml) was added, benzotriazol-1-yl-oxy-tris-pyrrolidino-phosphonium hexafluorophosphate (PyBOP; 2.6 mg, 0.005 mmol) dissolved in acetonitrile (0.25 ml), and N,N-diisopropylethylamine (1.7 μl, 0.010 mmol) were further added, and the mixture was shaken at room temperature for 5 hr. The gold film was sufficiently washed with acetonitrile and the condensation rate was determined (about 90%) by the method described in Production Example 12. In this way, a gold film bearing a hexaethylene glycol derivative as a hydrophilic spacer via alkanethiol derived from SAM was obtained (gold film bearing hexaethylene glycol derivative).

### Production Example 11

### Synthesis of Gold Film Bearing FK506 Derivative-Bound Hydrophilic Spacer (Gold Film+(PEG)₁—FK506)

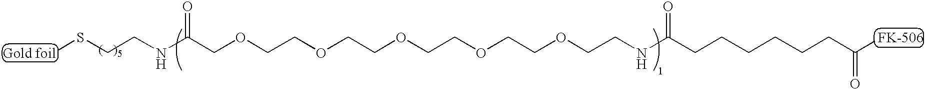

The gold film bearing a hexaethylene glycol derivative obtained in Production Example 10, and a mixture of 17-allyl-1,14-dihydroxy-12-{2-[4-(7-carboxy-heptanoyl-oxy)-3-methoxy-cyclohexyl]-1-methyl-vinyl}-23,25-dimethoxy-13,19,21,27-tetramethyl-11,28-dioxa-4-aza-tricyclo[22.3.1.0^{4,9}]octacos-18-ene-2,3,10,16-tetraone (4.8 mg, 0.005 mmol) prepared in Production Example 2, EDC.HCl (1.0 mg, 0.005 mmol), 1-hydroxybenzotriazole (HOBt; 0.7 mg, 0.005 mmol) and dimethylformamide (DMF; 0.5 ml) were stirred overnight at room temperature. After completion of the reaction, the gold film was sufficiently washed with DMF, immersed in a mixture of acetic acid (0.3 μl, 0.005 mmol) dissolved in DMF (0.25 ml), EDC.HCl (1.0 mg, 0.005 mmol), DMF (0.5 ml) containing HOBt (0.7 mg, 0.005 mmol) dissolved therein, and the mixture was stirred overnight at room temperature. The gold film was sufficiently washed with dimethylformamide (DMF) and acetonitrile to give a FK506-bound gold film bearing a hydrophilic spacer [gold film+(PEG)₁—FK506].

The number of HBA of the hydrophilic spacer intervening between the gold film and FK506 is 7, and the number of HBD thereof is 1. However, the number derived from alkanethiol moiety, which is derived from SAM, and group introduced into FK506 in advance are not counted in.

### Production Example 12

### Quantitation of Immobilization Amount of Low Molecular on Gold Thin Film by Quantitation of Fluorene Derivative

In Production Example 10, the 1.5 mM ethanol solution of (6-mercapto-hexyl)-carbamic acid 9H-fluoren-9-yl-methyl ester, in which the gold thin film was immersed overnight, was removed and the gold film was sufficiently washed with ethanol and acetonitrile, immersed in an acetonitrile solution containing 1 mL of 20% piperidine, and the mixture was shaken for 30 min. The acetonitrile solution was recovered, and the gold thin film was washed with 1 ml of acetonitrile. The recovered acetonitrile solution and the acetonitrile solution used for washing the gold thin film were combined and concentrated under reduced pressure. The residue was vacuum dried at 50° C. for 1 hr. After allowing to cool to room temperature, the fluorene derivative attached to the inside of the container was dissolved with 100 μL of acetonitrile, and 100 μL of milli Q water was further added. The solution was filtered, subjected to mass analysis with LC/MS of a fluorene derivative generated from Fmoc group by deprotection, and the fluorene derivative was quantitated from the obtained peak (M+1; 264) area. The amino group generated by deprotection was condensed with the hydrophilic spacer with an Fmoc group, which was obtained in Production Example 9, and deprotection with 20% piperidine and mass analysis of the produced fluorene derivative were performed, based on which the binding amount of hydrophilic spacer onto the gold thin film was determined.

### Reference Example 1

### Synthesis of FK506 Derivative-Bound (Alkanethiol Directly Bonded/No Hydrophilic Spacer) Gold Film

A gold film was treated with (6-mercapto-hexyl)-carbamic acid 9H-fluoren-9-yl-methyl ester according to the method described in Production Example 10 to immobilize alkanethiol on the gold film, and 17-allyl-1,14-dihydroxy-12-{2-[4-(7-carboxy-heptanoyl-oxy)-3-methoxy-cyclohexyl]-1-methyl-vinyl}-23,25-dimethoxy-13,19,21,27-tetramethyl-11,28-dioxa-4-aza-tricyclo[22.3.1.0^{4,9}]octacos-18-ene-2,3,10,16-tetraone was introduced thereinto by a method according to the description of Production Example 11.

### Reference Example 2

### Synthesis of Gold Film Bearing FK506 Derivative-Bound Dextran (Gold Film+Dextran—FK506)

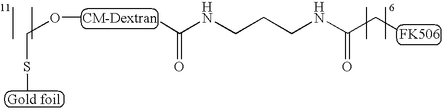

A gold film (Kojundo Chemical Laboratory Co., Ltd.; pure gold, purity 99.9% up, shape 10 mm×10 mm×0.01 mm (thickness)) (about 1 cm²) immersed in a Piranha solution (30% hydrogen peroxide: conc. sulfuric acid=1:4 mixed solution) for several hours was washed with milli Q water and ethanol. Using the gold film and according to the method described in a reference (J. Chem. Soc., Chem. Commun., 1526-1528, 1990), a gold film with carboxymethyldextran (CM-Dextran) was prepared. The obtained gold film with carboxymethyldextran and a mixture of 17-allyl-1,14-dihydroxy-12-{2-[4-(7-carboxy-heptanoyl-oxy)-3-methoxy-cyclohexyl]-1-methyl-vinyl}-23,25-dimethoxy-13,19,21,27-tetramethyl-11,28-dioxa-4-aza-tricyclo[22.3.1.0^{4,9}]octacos-18-ene-2,3,10,16-tetraone (9.6 mg, 0.01 mmol) prepared in Production Example 2, EDC.HCl (1.92 mg, 0.01 mol), HOBt (1.33 mg, 0.01 mmol), triethylamine (3.03 mg, 0.03 mol) and milli Q water (1 ml) were stirred overnight at room temperature. After completion of the reaction, the gold film was sufficiently washed with milli Q water, and immersed in a mixture of acetic acid (0.57 μl, 0.01 mmol), EDC.HCl (1.92 mg, 0.01 mmol), milli Q water (1 ml) containing HOBt (1.33 mg, 0.01 mmol) dissolved therein, and the mixture was stirred overnight at room temperature. The gold film was sufficiently washed with milli Q water to give a gold film bearing FK506-bound dextran [gold film+dextran—FK506].

### Production Example 13

### Synthesis of BIACORE Sensor Chip Bearing Hydrophilic Spacer: Sensor Chip (BIACORE; SIA Sensor Chip) Bearing Hexaethylene Glycol Derivative

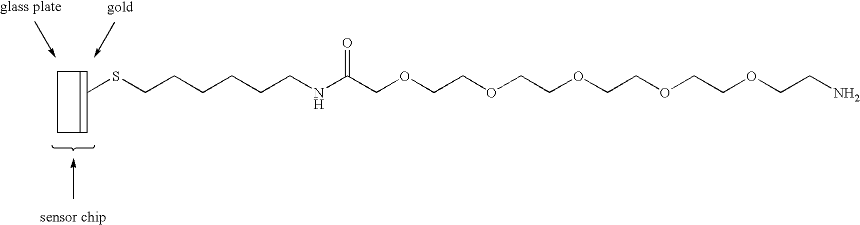

A sensor chip (BIACORE; SIA sensor chip) was immersed in a Piranha solution (30% hydrogen peroxide:con. sulfuric acid=1:4 mixed solution) for several hours and washed with milli Q water and ethanol. This was immersed overnight in a 1.5 mM ethanol solution (0.5 ml) of (6-mercapto-hexyl)-carbamic acid 9H-fluoren-9-yl-methyl ester to form SAM on the gold surface of the sensor chip. After completion of the reaction, the sensor chip was sufficiently washed with ethanol and acetonitrile, a mixed solution (1 ml) of piperidine/acetonitrile (1/4) was added and the mixture was shaken at room temperature for 30 min. The sensor chip was sufficiently washed with acetonitrile, {2-[2-(2-{2-[2-(9H-fluoren-9-yl-methoxycarbonylamino)-ethoxy]-ethoxy}-ethoxy)-ethoxy]-ethoxy}acetic acid (compound 8 obtained in Production Example 9; 12.5 mg, 0.024 mmol) dissolved in acetonitrile (0.25 ml) was added, benzotriazol-1-yl-oxy-tris-pyrrolidino-phosphonium hexafluorophosphate (PyBOP; 13 mg, 0.025 mmol) dissolved in acetonitrile (0.25 ml), and N,N-diisopropylethylamine (8.9 μl, 0.50 mmol) were further added, and the mixture was shaken overnight at room temperature. The reaction mixture was removed, and the sensor chip was washed with acetonitrile, and allowed to react overnight under the same conditions. After completion of the reaction, the sensor chip was sufficiently washed with acetonitrile and immersed in a mixed solution of acetic acid (0.3 μl, 0.005 mmol) dissolved in acetonitrile (0.25 ml), benzotriazol-1-yl-oxy-tris-pyrrolidino-phosphonium hexafluorophosphate (PyBOP; 2.6 mg, 0.005 mmol) dissolved in acetonitrile (0.25 ml), and N,N-diisopropylethylamine (1.7 μl, 0.010 mmol), and the mixture was is shaken at room temperature for 5 hr. The sensor chip was sufficiently washed with acetonitrile and treated with a mixed solution (1 ml) of piperidine/acetonitrile (1/4) as mentioned above. In this way, a BIACORE sensor chip bearing a hydrophilic spacer was obtained.

### Production Example 14

### Synthesis of Sensor Chip Bearing FK506 Derivative-Bound Hydrophilic Spacer (Sensor Chip+(PEG)₁—FK506)

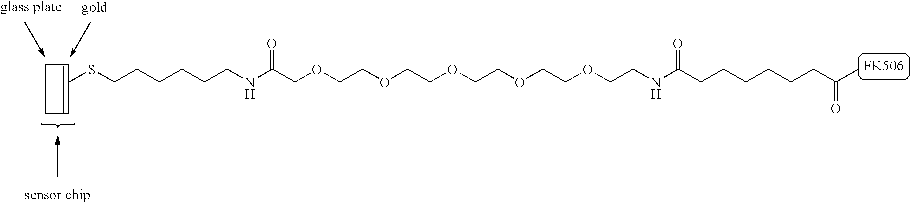

The sensor chip bearing a hexaethylene glycol derivative obtained in Production Example 13, and a mixture of 17-allyl-1,14-dihydroxy-12-{2-[4-(7-carboxy-heptanoyl-oxy)-3-methoxy-cyclohexyl]-1-methyl-vinyl}-23,25-dimethoxy-13,19,21,27-tetramethyl-11,28-dioxa-4-aza-tricyclo[22.3.1.0^{4,9}]octacos-18-ene-2,3,10,16-tetraone (4.8 mg, 0.005 mmol) prepared in Production Example 2, EDC.HCl (1.0 mg, 0.005 mmol), HOBt (0.7 mg, 0.005 mmol) and DMF (0.5 ml) were stirred overnight at room temperature. After completion of the reaction, the sensor chip was sufficiently washed with DMF, immersed in a mixture of acetic acid (0.3 μl, 0.005 mmol) dissolved in DMF (0.25 ml), EDC.HCl (1.0 mg, 0.005 mmol), DMF (0.5 ml) containing HOBt (0.7 mg, 0.005 mmol) dissolved therein, and the mixture was stirred overnight at room temperature. The sensor chip was sufficiently washed with DMF and acetonitrile to give a FK506-bound sensor chip bearing a hydrophilic spacer [sensor chip+(PEG)₁—FK506]. The number of HBA of the hydrophilic spacer intervening between the sensor chip having gold surface and FK506 is 7, and the number of HBD thereof is 1. However, the number derived from alkanethiol moiety which is derived from SAM, and group introduced into FK506 in advance are not counted in.

### Reference Example 3

### Synthesis of FK506 Derivative-Bound (Alkanethiol Directly Bonded/No Hydrophilic Spacer) Sensor Chip

A sensor chip was treated with (6-mercapto-hexyl)-carbamic acid 9H-fluoren-9-yl-methyl ester according to the method described in Production Example 10 to immobilize alkanethiol on the sensor chip, and 17-allyl-1,14-dihydroxy-12-{2-[4-(7-carboxy-heptanoyl-oxy)-3-methoxy-cyclohexyl]-1-methyl-vinyl}-23,25-dimethoxy-13,19,21,27-tetramethyl-11,28-dioxa-4-aza-tricyclo[22.3.1.0^{4,9}]octacos-18-ene-2,3,10,16-tetraone was introduced thereinto by a method according to the description of Production Example 11.

### Example 1

### (1) Preparation of Lysate

The rat brain (2.2 g) was mixed in a mixed fluid A (0.25M sucrose, 25 mM Tris buffer (pH 7.4), 22 ml) and prepared as a homogenate, which was then centrifuged at 9500 rpm for 10 minutes. The centrifugal supernatant was collected and further centrifuged at 50000 rpm for 30 minutes. The supernatant thus obtained was used as the lysate. Note that all experiments were conducted at 4° C. or on ice.

### (2) Binding Experiments

Using the above-mentioned various FK506-bound gold film (FK506 bound gold foils), a binding experiment to lysate was performed by the following steps. The lysate was diluted with mixed fluid A to ½ before use. Each one sheet (10 mm×10 mm×0.01 mm (thickness)) of various FK506-bound gold film was used.

As the FK506-bound gold film, a gold film bearing FK506 derivative-bound hydrophilic spacer of Production Example 11, into which a hexaethylene glycol derivative had been introduced, was used. As a Comparative Example, a gold film bearing FK506 (no hydrophilic spacer) of Reference Example 1 or a gold film bearing FK506 (dextran spacer) of Reference Example 2 was used.

The FK506-bound gold film and a lysate (1 ml) were gently shaken overnight at 4° C. Then, the supernatant was removed, and the resulting FK506-bound gold film was sufficiently washed 3 times with mixed fluid A, whereby each FK506-bound gold film was sufficiently washed.

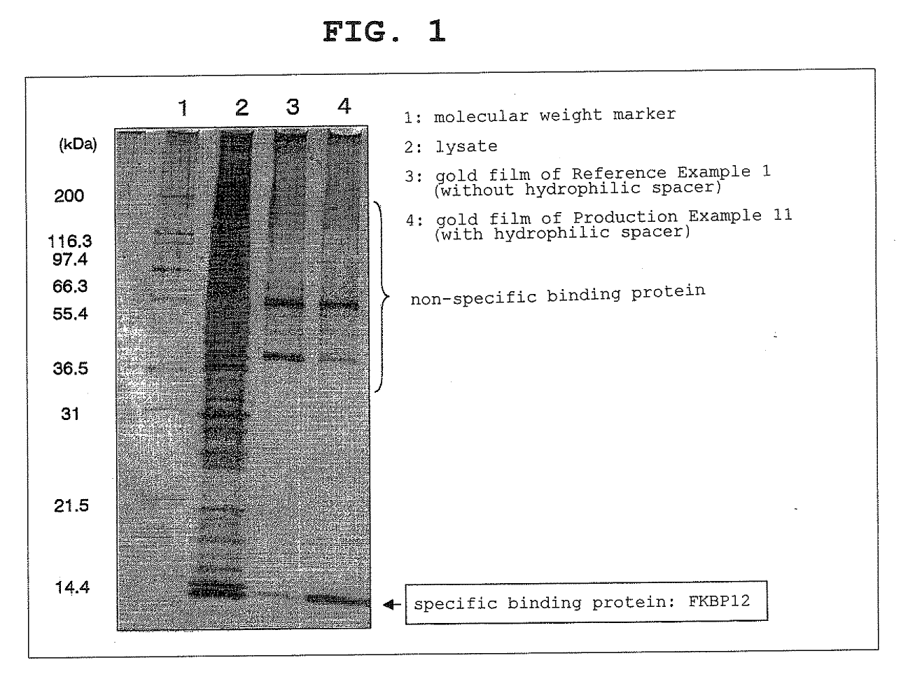

To the FK506-bound gold film thus obtained, 25 μl of a loading buffer for SDS-PAGE (Nacalai cat. NO=30566-22, sample buffer solution for electrophoresis with 2-ME (2-mercaptoethanol) (2×) for SDS PAGE) was added and pipetted. The sample fluid thus obtained was separated using a commercially available SDS gel (BioRad readyGel J, 15% SDS, cat. NO=161-J341), and the SDS gel was analyzed (FIG. 1). As a result, the one shown in FIG. 1, lane 4, with an introduced hydrophilic spacer showed a decrease or disappearance of the band intensity considered to be based on nonspecific interactions, and intensifying of the band (FKBP12) intensity considered to be based on specific interactions, as compared to the one shown in lane 3, which was free of a hydrophilic spacer. The results indicate that the introduction of a hydrophilic spacer suppressed nonspecific interactions and intensified specific interactions.

### Example 2

### (1) Preparation of Lysate

Performed according to Example 1.

### (2) Binding Experiment

Using the above-mentioned various FK506-bound sensor chips, a binding experiment to lysate was performed by the following steps. The lysate was diluted with mixed fluid A to ½ before use. Each one sheet of various FK506-bound sensor chips was used.

As the FK506-bound sensor chip, sensor chip bearing FK506-bound hydrophilic spacer of Production Example 14, into which a hexaethylene glycol derivative had been introduced, was used. As a Comparative Example, an FK506-bound sensor chip of Reference Example 3 was used.

The FK506-bound sensor chip and a lysate (1 ml) were gently shaken overnight at 4° C. Then, the supernatant was removed, and the resulting FK506-bound sensor chip was sufficiently washed 3 times with mixed fluid A, whereby each FK506-bound sensor chip surface was sufficiently washed.

To the gold surface of the FK506-bound sensor chip thus obtained, 25 μl of a loading buffer for SDS PAGE (Nacalai cat. NO=30566-22, sample buffer solution for electrophoresis with 2-ME (2-mercaptoethanol) (2×) for SDS PAGE) was added and pipetted. The sample fluid thus obtained was separated using a commercially available SDS gel (BioRad readyGel J, 15% SDS, cat. NO=161-J341), and the SDS gel was analyzed. As a result, the one with an introduced hydrophilic spacer showed a decrease or disappearance of the band intensity considered to be based on nonspecific interactions, and intensifying of the band (specifically, a band corresponding to FKBP12) intensity considered to be based on specific interactions. The results indicate that the introduction of a hydrophilic spacer suppressed nonspecific interactions and intensified specific interactions.

### INDUSTRIAL APPLICABILITY

Introduction of a hydrophilic spacer in between a metal surface and a ligand to be the examination target, during immobilization of the ligand on the surface of metal as a solid phase carrier enables reduction of hydrophobic property of the metal surface, and suppression of nonspecific intermolecular interactions. Simultaneously, specific intermolecular interactions can be intensified.

In a study including measurement of interactions of low molecule—low molecule, low molecule—high molecule, high molecule—high molecule, or purification of an object target based on the interaction, it is possible to artificially suppress nonspecific interactions or intensify specific interactions, by the technique of the present invention. To be specific, the present technique facilitates a study including immobilizing one molecule of low molecule—high molecule, low molecule—low molecule, high molecule—high molecule on a solid phase carrier and measuring interactions thereof, or purifying an object target based on the interaction. Such achievement is widely applicable to life science in general, particularly drug discovery research, post-genomic research, proteomics, chemical genomics, chemical proteomics and the like.

1. A method of suppressing a nonspecific interaction between a ligand and/or a metal surface and a molecule other than a target molecule, which comprises, in a process of immobilizing the ligand onto the metal surface and analyzing a specific interaction on the metal surface between the ligand and the target molecule thereof, a treatment to reduce the hydrophobic property of the metal surface.

2. A method of intensifying a specific interaction between a ligand and a target molecule, which comprises, in a process of immobilizing the ligand onto a metal surface and analyzing a specific interaction on the metal surface between the ligand and the target molecule of the ligand, a treatment to reduce the hydrophobic property of the metal surface.

3. A method of suppressing a nonspecific interaction between a ligand and/or a metal surface and a molecule other than a target molecule and intensifying a specific interaction between the ligand and the target molecule, which comprises, in a process of immobilizing the ligand onto the metal surface and analyzing a specific interaction on the metal surface between the ligand and the target molecule of the ligand, a treatment to reduce the hydrophobic property of the metal surface.

4. A method of suppressing a nonspecific interaction between a ligand and/or a metal surface and a molecule other than a target molecule, which comprises, in a process of immobilizing the ligand onto the metal surface and selecting a target molecule using a specific interaction on the metal surface between the ligand and the target molecule thereof, a treatment to reduce the hydrophobic property of the metal surface.

5. A method of intensifying a specific interaction between a ligand and a target molecule, which comprises, in a process of immobilizing the ligand onto the metal surface and selecting the target molecule using a specific interaction on the metal surface between the ligand and the target molecule thereof, a treatment to reduce the hydrophobic property of the metal surface.

6. A method of suppressing a nonspecific interaction between a ligand and/or a metal surface and a molecule other than a target molecule and intensifying a specific interaction between the ligand and the target molecule, which comprises, in a process of immobilizing the ligand onto the metal surface and selecting the target molecule using a specific interaction on the metal surface between the ligand and the target molecule thereof, a treatment to reduce the hydrophobic property of the metal surface.

7. The method of claim 1, wherein the treatment to reduce the hydrophobic property of the metal surface is to introduce, at the time of immobilization of the ligand onto the metal surface, a hydrophilic spacer therebetween.

8. The method of claim 7, wherein the hydrophilic spacer has at least any of the following characteristics while in a state bound to the metal surface and the ligand:
(i) the number of hydrogen bond acceptor is 6 or more,
(ii) the number of hydrogen bond donor is 5 or more,
(iii) the total number of hydrogen bond acceptor and hydrogen bond donor is 9 or more.

9. The method of claim 8, wherein said hydrophilic spacer further has one or more carbonyl groups in the molecule thereof.

10. The method of claim 8, further characterized in that said hydrophilic spacer does not have a functional group that becomes positively or negatively charged in an aqueous solution.

11. A method of immobilizing a ligand onto a metal surface and analyzing a specific interaction on the metal surface between the ligand and a target molecule thereof which comprises suppressing a nonspecific interaction between the ligand and/or the metal surface and a molecule other than the target molecule by a treatment to reduce the hydrophobic property of the metal surface.

12. A method of immobilizing a ligand onto a metal surface and analyzing a specific interaction on the metal surface between the ligand and a target molecule thereof, which comprises intensifying a specific interaction between the ligand and the target molecule by a treatment to reduce the hydrophobic property of the metal surface.

13. A method of immobilizing a ligand onto a metal surface and analyzing a specific interaction on the metal surface between the ligand and a target molecule thereof, which comprises suppressing a nonspecific interaction between the ligand and/or the metal surface and a molecule other than the target molecule and intensifying a specific interaction between the ligand and the target molecule, by a treatment to reduce the hydrophobic property of the metal surface.

14. A method of immobilizing a ligand onto a metal surface, and selecting a target molecule using a specific interaction on the metal surface between the ligand and the target molecule thereof, which comprises suppressing a nonspecific interaction between the ligand and/or the metal surface and a molecule other than the target molecule by a treatment to reduce the hydrophobic property of the metal surface.

15. A method of immobilizing a ligand onto a metal surface, and selecting a target molecule using a specific interaction on the metal surface between the ligand and the target molecule thereof, which comprises intensifying a specific interaction between the ligand and the target molecule by a treatment to reduce the hydrophobic property of the metal surface.

16. A method of immobilizing a ligand onto a metal surface, and selecting a target molecule using a specific interaction on the metal surface between the ligand and the target molecule thereof, which comprises suppressing a nonspecific interaction between the ligand and/or the metal surface and a molecule other than the target molecule and intensifying a specific interaction between the ligand and the target molecule, by a treatment to reduce the hydrophobic property of the metal surface.

17. The method of claim 11, wherein the treatment to reduce the hydrophobic property of the metal surface is to introduce, at the time of immobilization of the ligand onto the metal surface, a hydrophilic spacer therebetween.

18. The method of claim 17, wherein the hydrophilic spacer has at least any of the following characteristics while in a state bound to the metal surface and the ligand:
(i) the number of hydrogen bond acceptor is 6 or more,
(ii) the number of hydrogen bond donor is 5 or more,
(iii) the total number of hydrogen bond acceptor and hydrogen bond donor is 9 or more.

19. The method of claim 18, wherein said hydrophilic spacer further has one or more carbonyl groups in the molecule thereof.

20. The method of claim 18, further characterized in that said hydrophilic spacer does not have a functional group that becomes positively or negatively charged in an aqueous solution.

21. A screening method for a target molecule having a specific interaction with a ligand, comprising at least the following steps:
(i) immobilizing the ligand onto a metal surface via a hydrophilic spacer,
(ii) contacting a sample that contains or does not contain the target molecule with the metal surface with the ligand immobilized thereon obtained in (i) above,
(iii) identifying and analyzing a molecule that has exhibited or has not exhibited a specific interaction with the ligand, and
(iv) judging a molecule that exhibits a specific interaction with the ligand as the target molecule on the basis of the analytical results obtained in (iii) above.

22. The method of claim 21, wherein the hydrophilic spacer has at least any of the following characteristics while in a state bound to the metal surface and the ligand:
(i) the number of hydrogen bond acceptor is 6 or more,
(ii) the number of hydrogen bond donor is 5 or more,
(iii) the total number of hydrogen bond acceptor and hydrogen bond donor is 9 or more.

23. The method of claim 22, wherein said hydrophilic spacer further has one or more carbonyl groups in the molecule thereof.

24. The method of claim 22, further characterized in that said hydrophilic spacer does not have a functional group that becomes positively or negatively charged in an aqueous solution.

25. The method of claim 7, wherein the hydrophilic spacer has at least one partial structure represented by any one formula selected from the group consisting of Formulas (Ia)-(Ie) below: 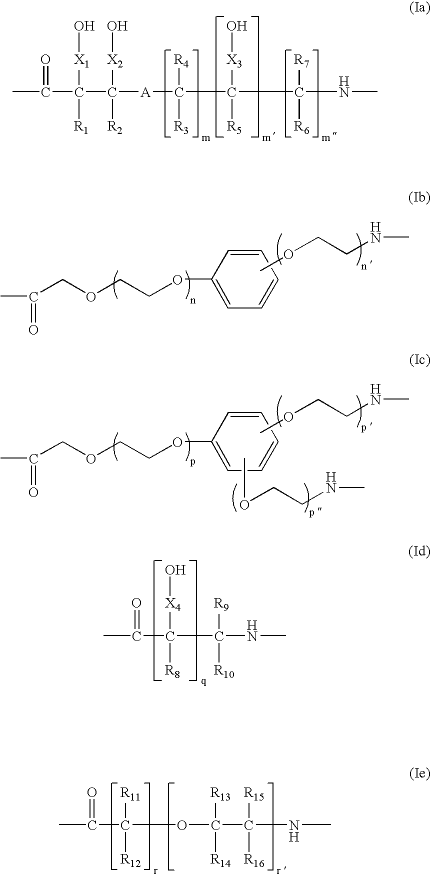
(In Formula (Ia),
A is an appropriate joining group,
X₁-X₃ are the same or different and each is a single bond or a methylene group that may be substituted by a linear or branched alkyl group having 1-3 carbon atoms,
R₁-R₇ are the same or different and each is a hydrogen atom, a linear or branched alkyl group having 1-3 carbon atoms, —CH₂OH or a hydroxyl group,
m is an integer of 0-2, m′ is an integer of 0-10, m″ is an integer of 0-2,
when a plurality of R₃-R₇ units exist, they may be the same or different, when a plurality of X₃ units exist, they may be are the same or different;
in Formula (Ib),
n and n′ are the same or different and each is an integer of 1-1000;
in Formula (Ic),
p, p′ and p″ are the same or different and each is an integer of 1-1000;
in Formula (Id),
X₄ is a single bond or a methylene group that may be substituted by a linear or branched alkyl group having 1-3 carbon atoms,
R₈-R₁₀ are the same or different and each is a hydrogen atom, a linear or branched alkyl group having 1-3 carbon atoms, —CH₂OH or a hydroxyl group,
q is an integer of 1-7,
when a plurality of R₈ units exist, they may be the same or different, when a plurality of X₄ units exist, they may be the same or different;
in Formula (Ie),
R₁₁-R₁₆ are the same or different and each is a hydrogen atom, a linear or branched alkyl group having 1-3 carbon atoms, —CH₂OH or a hydroxyl group,
r is an integer of 1-10, r′ is an integer of 1-50,
when a plurality of R₁₁-R₁₆ units exist, they may be the same or different).

26. The method of claim 25, wherein the hydrophilic spacer has two or more partial structures represented by any one formula selected from the group consisting of Formulas (Ia)-(Ie).

27. A solid phase carrier with a ligand immobilized thereon, which is a metal and has a hydrophilic spacer between the metal and the ligand.

28. The solid phase carrier of claim 27, wherein the hydrophilic spacer has at least one partial structure represented by any one formula selected from the group consisting of Formulas (Ia)-(Ie) below: 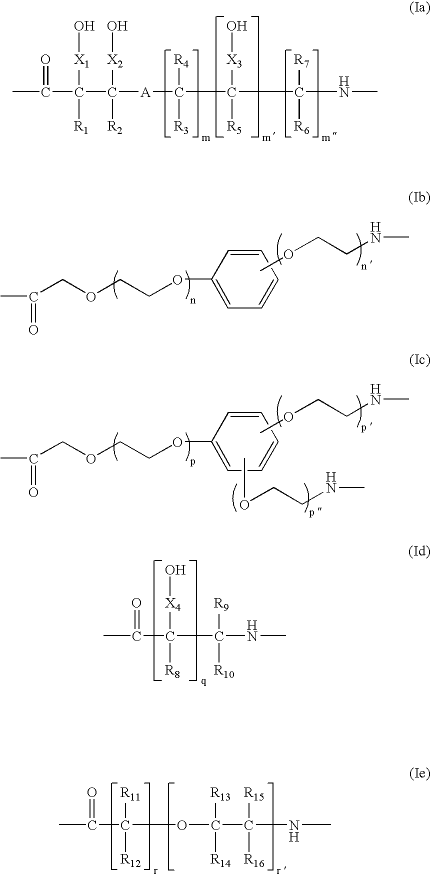
(In Formula (Ia),
A is an appropriate joining group,
X₁-X₃ are the same or different and each is a single bond or a methylene group that may be substituted by a linear or branched alkyl group having 1-3 carbon atoms,
R₁-R₇ are the same or different and each is a hydrogen atom, a linear or branched alkyl group having 1-3 carbon atoms, —CH₂OH or a hydroxyl group,
m is an integer of 0-2, m′ is an integer of 0-10, m″ is an integer of 0-2,
when a plurality of R₃-R₇ units exist, they may be the same or different, when a plurality of X₃ units exist, they may be are the same or different;
in Formula (Ib),
n and n′ are the same or different and each is an integer of 1-1000;
in Formula (Ic),
p, p′ and p″ are the same or different and each is an integer of 1-1000;
in Formula (Id),
X₄ is a single bond or a methylene group that may be substituted by a linear or branched alkyl group having 1-3 carbon atoms,
R₈-R₁₀ are the same or different and each is a hydrogen atom, a linear or branched alkyl group having 1-3 carbon atoms, —CH₂OH or a hydroxyl group,
q is an integer of 1-7,
when a plurality of R₈ units exist, they may be the same or different, when a plurality of X₄ units exist, they may be the same or different;
in Formula (Ie),
R₁₁-R₁₆ are the same or different and each is a hydrogen atom, a linear or branched alkyl group having 1-3 carbon atoms, —CH₂OH or a hydroxyl group,
r is an integer of 1-10, r′ is an integer of 1-50,
when a plurality of R₁₁-R₁₆ units exist, they may be the same or different).

29. The solid phase carrier of claim 27, wherein the metal is gold.

30. A method of confirming introduction of a hydrophilic spacer between a ligand and a metal surface, which comprises, in a step of introducing the hydrophilic spacer between them during immobilization of the ligand onto the metal surface, detecting a leaving group produced by elimination of a protecting group derived from the hydrophilic spacer.

31. The method of claim 30, wherein the leaving group is detected by mass analysis.

32. The method of claim 30, wherein the protecting group is a 9-fluorenylmethyloxycarbonyl group.
